(19) United States
(12) Patent Application Publication
(10) Pub. No.: US 2005/0025728 A1
(43) Pub. Date: Feb. 3, 2005

(54) Cosmetic compositions and contrast cards for characterizing them

(75) Inventors: Jean De Rigal, Gressy (FR); Nicolas Soistier, Le Kremlin Bicetre (FR); Hojung Lee, Paris (FR)

Correspondence Address: OLIFF & BERRIDGE, PLC, P.O. BOX 19928, ALEXANDRIA, VA, 22320, US

(73) Assignee: L'Oreal, Paris (FR)

(21) Appl. No.: 10/743,455

(22) Filed: Dec. 23, 2003

Related U.S. Application Data
(60) Provisional application No. 60/439,798, filed on Jan. 14, 2003.

(30) Foreign Application Priority Data
Dec. 24, 2002 (FR) FR 02 16653

(51) Int. Cl. A61K 7/021

(52) U.S. Cl. 424/63

(57) ABSTRACT

A contrast card comprising at least two colored zones corresponding respectively to the average color of at least two regions of the face of a panel of individuals.

This application claims the benefit of U.S. Provisional Application No. 60/439,798 filed on Jan. 14, 2003, the disclosure of which is incorporated by reference herein.

### BACKGROUND OF THE INVENTION

The present invention relates to cosmetic compositions to be applied to the skin and more particularly, but not exclusively, compositions of foundation type, intended for dark skins, including black and mixed-race skins.

The expression “composition of foundation type” denotes a makeup composition for human skin. It may be a foundation to be applied to the face or the neck, a concealer product, a complexion-correcting product, a tinted cream or makeup base for the face or a makeup composition for the body.

The term “dark skins” denotes skins whose mean lightness L* measured on the forehead, the cheekbones and the chin, in the CIE 1976 colorimetric space, is less than 60, and especially less than 55. The saturation C* may be, for example, between 8 and 30, in particular between 10 and 30 or even between 12 and 28. The hue angle values h may be, for example, between about 38° and about 65° and especially between 46° and 63°, for example between 46° and 54°. The lightness values L* may be less than or equal to 50, or even 45 or 40 for the darkest skins, while at the same time remaining above 30 for the majority of skins. Dark skins are encountered, for example, among the African, Afro-American, Hispano-American, Indian and maghrebine populations.

It is known practice, in order to lighten such skins, to use products containing bleaching active agents, some of which are relatively aggressive, for instance hydroquinone, and require prolonged treatment to produce a result.

Another possibility is to use a foundation that is paler than its complexion. However, dark skins show a wide variety of complexions, thus making it difficult to select a suitable foundation. Furthermore, the makeup result may not appear as natural as desirable, with a greyish and dull effect in particular.

### SUMMARY OF THE INVENTION

The invention is directed towards proposing a cosmetic composition capable of lightening a dark skin while at the same time giving a natural makeup result.

According to one of its aspects among others, one subject of the invention is thus a composition of foundation type, comprising, in a physiologically acceptable medium, at least one coloring agent, the composition being able to be characterized in that it is capable, when it is applied to a dark skin, of producing a positive lightness variation ΔL* between 0.5 and 4 and a positive saturation variation ΔC* of between 0.5 and 4.

Such variations in lightness L* and in saturation C* make it possible to lighten the skin without resulting in a greyish or dull effect.

In particular embodiment examples of the invention, the lightness variation ΔL* and saturation variation ΔC* are between 0.5 and 3, for example between 0.5 and 2.5. With such values, the result is particularly satisfactory.

The composition may also advantageously be suitable for producing a negative variation in the coxellographic index Δiₒₒₓ of less than or equal to −0.4, preferably less than or equal to −0.5 and more preferably less than or equal to −1, for an individual having before makeup a coxellographic index i_{cox} of greater than or equal to 5. This makes it possible to lighten the skin while at the same time making the color of the face more uniform, in order, for example, to conceal hyperpigmented regions, without excessively degrading the natural appearance of the skin.

The term “coxellographic index i_{cox}” denotes the product of the standard deviations σₐ*×σ_{b}*×σ_{L}* for all of the zones observed, as will be specified hereinbelow.

Examples of lightening compositions have the following calorimetric characteristics:
a lightness L* of greater than or equal to 34, a position b* on the blue/yellow axis of between 28 and 42 and a saturation C* of between 31 and 52, better still a lightness L* of between 34 and 41, a value b* of between 33 and 42 and a saturation C* of between 37 and 46, such a composition possibly being used to lighten a skin with a complexion defined by a lightness L* of less than 42, especially between 35 and 42, and a saturation of less than 21, and especially between 13 and 20,
a lightness L* of greater than or equal to 34, a position b* on the blue/yellow axis of between 23 and 39 and a saturation C* of between 27 and 47, better still a lightness L* of between 34 and 48, a position b* of between 27 and 35 and a saturation C* of between 31 and 41,
a lightness L* of greater than or equal to 34, a position b* on the blue/yellow axis of between 19 and 33 and a saturation C* of between 23 and 40, and especially a lightness L* of between 34 and 42, a position b* of between 23 and 29 and a saturation C* of between 28 and 35,
a lightness L* of greater than or equal to 39, a position b* on the blue/yellow axis of between 23 and 39 and a saturation C* of between 38 and 41, and better still a lightness L* of between 39 and 48, a position b* of between 28 and 35 and a saturation C* of between 33 and 41,
a lightness L* of greater than or equal to 51, a position b* on the blue/yellow axis of between 14 and 25 and a saturation C* of between 18 and 31, and better still a lightness L* of between 51 and 63, a position b* of between 14 and 25 and a saturation C of between 22 and 28, such a composition possibly being used to lighten a skin with a lightness of between 48 and 54 and a saturation of between 24 and 28,
a lightness L* of greater than or equal to 34, a position b* on the blue/yellow axis of between 11 and 20 and a saturation C* of between 17 and 29, and better still a lightness L* of between 34 and 48, a position b* of between 14 and 17 and a saturation C* of between 20 and 26.

A subject of the invention is also, independently of or in combination with the foregoing text, according to another of its aspects, a composition of foundation type comprising, in a physiologically acceptable medium, at least one coloring agent and reflective particles, the said composition having a hue in the color range extending from pink-beige to orange-brown.

A subject of the invention is also, independently of or in combination with the foregoing text, according to another of its aspects, a composition of foundation type comprising, in a physiologically acceptable medium, at least one coloring agent and reflective particles, the said composition having a hue angle h ranging from 40 to 70°, and especially 50° to 70°, and a saturation C* ranging from 20 to 50.

A subject of the invention is also, independently of or in combination with the foregoing text, according to another of its aspects, a composition of foundation type, comprising, in a physiologically acceptable medium, at least one coloring agent and reflective particles, the said composition having a reflectance ranging from 10 to 45% in the range from 600 to 680 nm.

A subject of the invention is also, independently of or in combination with the foregoing text, according to another of its aspects, a product of foundation type especially for making up the skin, in particular dark skins, comprising at least one first and one second composition, each in a container, the first composition comprising, in a first physiologically acceptable medium, at least one coloring agent, and the second composition comprising, in a second physiologically acceptable medium, at least reflective particles.

A subject of the present invention is also, according to another of its aspects, a method for making up dark skin, comprising the application to the skin of a product containing two compositions as defined above. This method may comprise the application of a first coat of one of the two compositions, known as the base composition, following by the application over at least a portion of the first coat of a second coat of the other composition, known as the surface composition.

The makeup thus obtained is a two-coat makeup. The order of superposition and/or the mode of superposition of the two compositions, i.e or total or partial, may moreover, where appropriate, give additional aesthetic effects.

A subject of the invention is also, according to another of its aspects, the use of one of the compositions as defined above to lighten dark skin.

A subject of the invention is also, according to another of its aspects, a method for marketing a composition intended to lighten dark skin, which comprises the step consisting in taking into account, during the marketing of this composition, the color of the skin for which the composition is preferably intended. Such a method may comprise the presentation, by any means, of the skin colors for which the composition is intended, for example by using a colored indicator given on a container or packaging containing the composition. Reference may also be made on the packaging or container to an ethnic group in which the type of skin color for which the composition is intended is frequently found.

A subject of the invention is also a method in which a colorimetric characteristic of a dark skin is measured, especially its lightness and/or its saturation, and in which a composition suitable for lightening this skin is selected from the measured colorimetric characteristic, especially a composition producing variations in lightness and saturation and optionally in coxellographic index, as mentioned above.

Independently of or in combination with the foregoing text, a subject of the invention is also a composition of foundation type comprising, in a physiologically acceptable medium, at least one coloring agent, this composition being able to be characterized in that it is capable of having a homogenization power 1/ΔE₁ and a covering power 1/ΔE₂ below, when it is applied, according to the value of its lightness L*, to one of the following contrast cards:

when the composition has a lightness L* of between 30 and 40, when it is applied to a contrast card with five zones each respectively having as calorimetric coordinates, to within 15%, or even 12.5%, better still 10%, or even 7.5%, or even 5%, for L* and h, and to within 25%, better still 20%, even better still 15%, or even 10%, or even 5%, for C*,

| | | | |
|---|---|---|---|
| first zone (B11+): | L* = 36.7 | C* = 19.81 | h = 47.34°, |
| second zone (B11): | L* = 38.43 | C* = 21.76 | h = 46.51°, |
| third zone (B12): | L* = 35.66 | C* = 19.78 | h = 46.32°, |
| fourth zone (B12+): | L* = 32.98 | C* = 17.29 | h = 44.64°, |
| fifth zone (XXX): | L* = 29.63 | C* = 15.06 | h = 40.34°, |

the composition is capable of having a homogenization power 1/ΔE_{1 mean} of between about 1.1 and about 6.7, better still between about 1.25 and about 5, and a covering power 1/ΔE₂ of between about 0.25 and about 0.84;

when the composition has a lightness of between 40 and 50, when it is applied to a contrast card with five zones each respectively having as colorimetric coordinates, to within 15%, better still 12.5%, even better still 10%, or even 7.5%, or even 5% for L* and h, and to within 25%, better still 20%, even better still 15%, or even 10%, or 5%, for C*,

| | | | |
|---|---|---|---|
| first zone (C9): | L* = 45.04 | C* = 25.18 | h = 53.27°, |
| second zone (B12): | L* = 35.66 | C* = 19.78 | h = 46.32°, |
| third zone (C11): | L* = 38.73 | C* = 21.94 | h = 50.18°, |
| fourth zone (C10): | L* = 42.19 | C* = 24.18 | h = 51.94°, |
| fifth zone (C8): | L* = 48.06 | C* = 25.97 | h = 53.09°, |

the composition is capable of having a homogenization power 1/ΔE_{1 mean} of between about 1.1 and about 1.54 and better still between about 1.1 and about 1.43, and a covering power 1/ΔE₂ of between about 1/9 and ⅕ and better still between about ⅛ and about 0.15,

when the composition has a lightness of between 50 and 60, when it is applied to a contrast card with five zones each respectively having as colorimetric coordinates, to within 15%, better still 12.5%, even better still 10%, or even 7.5%, or even 5% for L* and h, and to within 25%, better still 20%, even better still 15%, or even 10% or 5%, for C*,

| | | | |
|---|---|---|---|
| first zone (D6): | L* = 54.08 | C* = 26.70 | h = 57.35°, |
| second zone (C11): | L* = 38.73 | C* = 21.94 | h = 50.18°, |
| third zone (D8): | L* = 47.94 | C* = 26.18 | h = 56.82°, |
| fourth zone (D7): | L* = 51.79 | C* = 27.21 | h = 57.09°, |
| fifth zone (D5): | L* = 57.61 | C* = 26.22 | h = 55.09°, |

the composition is capable of having a homogenization power 1/ΔE_{1 mean} of between about 0.8 and about 1.25 and a covering power 1/ΔE₂ of between about 1/7 and ⅓, the homogenization power and covering power being defined later.

Independently of or in combination with the foregoing text, a subject of the invention is also a composition of foundation type comprising, in a physiologically acceptable medium, at least one coloring agent, this composition being able to be characterized in that it is capable of having, when it is applied to a contrast card with five zones each respectively having as colorimetric coordinates, to within 5%:

| | | | |
|---|---|---|---|
| first zone (Z1): | L* = 48.38 | a* = 7.99 | b* = 3.85 |
| second zone (Z2): | L* = 46.67 | a* = 6.78 | b* = 3.25 |
| third zone (Z3): | L* = 44.5 | a* = 6.76 | b* = 3.1 |
| fourth zone (Z4): | L* = 42.72 | a* = 4.12 | b* = 2.57 |
| fifth zone (Z5): | L* = 44.41 | a* = 6.57 | b* = 3.93 |

and a sixth zone (Z6) having as colorimetric coordinates

| | | |
|---|---|---|
| L* = 52.26 | a* = 9.11 | b* = 5.81, |

a homogenization power 1/ΔE_{1 mean} of between ¼ and 1 and better still between ⅓ and ½, and a covering power 1/ΔE₂ of between 1/25 and 1/7 and better still between 1/21 and 1/10, the homogenization power and covering power being defined later.

Compositions with lightening effects that are suitable for certain complexions at least may have, for example, when they are applied to the above-mentioned contrast card comprising six zones (Z1, Z2, Z3, Z4, Z5 and Z6):
a homogenization power 1/ΔE_{1 means} of between 1/1.6 and ½ and a covering power 1/ΔE₂ of between 1/12 and 1/15,
a homogenization power 1/ΔE_{1 mean} of between 1/1.8 and ½, and a covering power 1/ΔE₂ of between 1/13 and 1/17,
a homogenization power 1/ΔE₁ mean of between 1/1.6 and 1/2.1 and a covering power 1/ΔE₂ of between 1/12 and 1/16,
a homogenization power 1/ΔE_{1 means} of between 1/2.6 and ⅓ and a covering power 1/ΔE₂ of between 1/16 and 1/21, or
a homogenization power 1/ΔE_{1 mean} of between 1/1.7 and ½, and a covering power 1/ΔE₂ of between 1/9 and 1/13.

A subject of the invention is also, independently of or in combination with the foregoing text, a contrast card which may be characterized in that it comprises at least two colored zones corresponding, respectively, to the mean color of at least two regions of the face of a panel of individuals.

The card may also comprise another colored zone corresponding to the mean color of a region of the body located other than on the face.

All the colored zones may advantageously be prepared so as to have substantially the same color under two different illuminants.

In one particular embodiment, the card comprises at least three colored zones corresponding, respectively, to the mean color of the forehead, of a bag under the eyes and of the region between the top lip and the nose of the individuals of the panel. The card may also comprise two colored zones corresponding to the color of skin marks of individuals of the panel.

The card may also comprise a white zone, or even also a black zone.

A subject of the invention is also, according to another of its aspects, a method for determining at least one colorimetric characteristic of a composition, comprising the following steps:
coating a contrast card from among those defined above with a coat of a composition,
measuring the color of the said zones of the card via the composition,
determining at least one colorimetric characteristic of the composition, especially the homogenization power and/or the covering power, as a function of color differences measured between the said zones.

The composition, especially when it is liquid, may be applied to a transparent support placed on the card, thus avoiding impairment of the card. Such a support may advantageously be adhesive when the composition is pulverulent.

A subject of the invention is also, according to another of its aspects, a method for manufacturing a contrast card for evaluating at least one calorimetric characteristic of a composition, comprising the following steps:
selecting a panel of individuals having the same typology of skin:
   for each individual of the panel,
      measuring the color of at least one region of the body located other than on the face,
      measuring the color of at least one region of the face,
calculating a mean color for each region,
reproducing, by printing, the mean colors thus calculated on a contrast card.

A subject of the invention is also, according to another of its aspects,
a method for manufacturing a contrast card to evaluate at least one colorimetric characteristic of a composition, comprising the following steps:
   selecting a panel of individuals having the same typology of skin:
      for each individual of the panel,
      measuring the color of at least two different regions of the face,
calculating a mean color for each region,
reproducing, by printing, the mean colors thus calculated on a contrast card.

For at least one individual of the panel, and preferably for all the individuals of the panel, the color of at least three different regions of the face, especially the forehead, the region between the lips and the nose and the shadows under the eyes, may be measured.

A subject of the invention is also, according to another of its aspects, a method for manufacturing a composition to be applied to skin having a given typology, comprising the following steps:
selecting at least one coloring agent for the composition using a contrast card as defined above,
manufacturing the composition with this coloring agent.

### BRIEF DESCRIPTION OF THE DRAWINGS

The invention may be understood more clearly on reading the detailed description that follows, of non-limiting examples of its implementation, and on examining the attached drawing, in which:

FIG. 1 shows diagrammatically and partially an acquisition device that may be used to take calorimetric measurements,

FIGS. 2 and 3 show diagrammatically a face placed in the aperture of the sphere of the device of FIG. 1, on which calorimetric measurements are taken,

FIG. 4 shows examples of contrast cards that may be used to determine colorimetric characteristics of compositions for lightening the skin,

FIGS. 5 and 6 show other examples of contrast cards that may be used to determine colorimetric characteristics of compositions for lightening the skin,

FIG. 7 shows reflectance spectra of compositions,

FIG. 8 shows diagrammatically an example of packaging comprising at least one colored indicator, and

Table I shows examples of spectral reflectance values in percentages as a function of the wavelength and of the zones.

### DETAILED DESCRIPTION OF THE PREFERRED EMBODIMENTS

Measurements of L* and C* (in the CIE 1976 space)

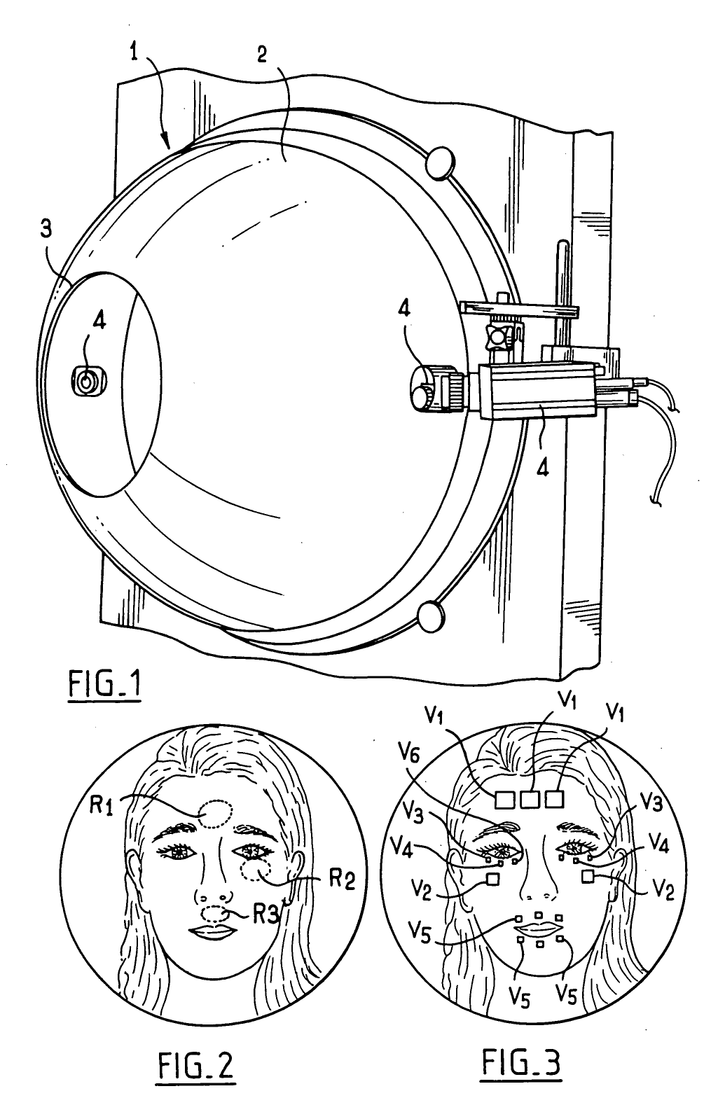

The lightness L* and the saturation C* may be measured before and after applying makeup, using an acquisition device 1 shown schematically and partially in FIG. 1, comprising a sphere 2 inside which are arranged several sources of white light, not apparent, such that the light reaching its aperture 3 is as homogeneous as possible.

The face of the individual whose color it is desired to measure is placed in this aperture 3 and images are acquired using color cameras 4 or digital photography devices.

Prior to acquiring the images, the acquisition line is calibrated by placing reference calorimetric standards in the field of the cameras, for example according to the method described in patent U.S. Pat. No. 6,362,849, the content of which is incorporated in the present patent application by reference.

The color measurements may be taken, for example, on three regions of the face, ie, in the example in FIG. 2, a first region R1 on the forehead, a second region R2 corresponding to shadows under the eyes and a third region R3 between the nose and the lips.

The color is measured before applying makeup to-the precleansed skin, and then after applying makeup, for example 15 minutes after the end of this application of makeup.

The makeup compositions are, in the example under consideration, liquid foundations applied in the usual manner directly to the skin, without prior application of a base, at a rate of from 0.5 to 1 mg approximately per cm and preferably between 0.7 and 0.8 mg/cm². Needless to say, the invention is not limited to compositions in the form of liquids, and also applies to compositions in the form of sticks or free or compacted powders. The invention also applies to compositions distributed in spray form.

Each measurement is taken on an image on which the zones whose color it is desired to determine appear, the regions R1, R2 or R3 each corresponding, for example, to more than 100 pixels of the image, for example about 250 pixels. For each pixel, the coordinates L*, a* and b* are determined before and after applying makeup.

Calculation of the Coxellographic Index i_{cox}

The standard deviation σ may be calculated for each parameter L*, a* and b* for all of the pixels of the three regions R1, R2 and R3, ie, for example, for about 750 pixels, before and after applying makeup.

The coxellographic index i_{cox} is defined as being the product of the three standard deviations σₐ*, σ_{b}* and σ_{L}* :i_{cox}=×σ_{b}*×σ_{L}*

The more uniform the color of the skin, the lower the coxellographic index i_{cox}.

By comparing the lightness L* and saturation C* before and after applying makup to the regions R1, R2 and R3, the makeup could be considered as being satisfactory when the variations ΔL* of the lightness and ΔC* of the saturation were between 0.5 and 4 and better still between 0.5 and 2.5.

For individuals whose skin is non-homogeneous and has a coxellographic index i_{cox}≧5, a homogenization effect could be adopted when the application of the makeup resulted in a variation of the coxellographic index i_{cox}<−0.4.

Contrast Cards

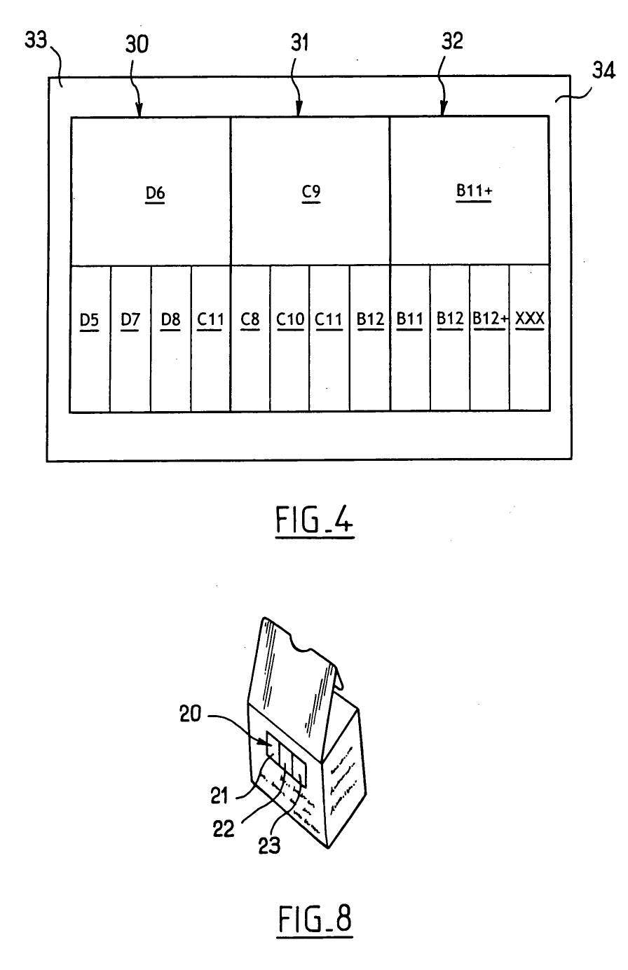

Contrast cards may advantageously be used, as illustrated in FIG. 4, to demonstrate especially the homogenization power and covering power of a composition.

In this figure, three contrast cards 30, 31 and 32 combined on the same support 33 are shown. It is not a departure from the context of the present invention if the contrast cards 30, 31 and 32 are produced on separate supports.

The support 33 comprises a white border 34 extending around the contrast cards 30, 31 and 32, which are side by side.

The contrast card 30 comprises a colored zone D6, for example a square zone, and four colored zones D5, D7, D8 and C11, for example rectangular zones, arranged side by side and adjacent to the colored zone D6 along one of their small sides.

The contrast card 31 comprises a square colored zone C9 and four rectangular colored zones C8, C10, C11 and B12.

Similarly, the contrast card 32 comprises a square colored zone B11+ and four rectangular colored zones B11, B12, B12+ and XXX.

The various zones D5 to D8, C8 to C11, B11, B11+, B12, B12+ and XXX are preferably made by printing inks chosen such that these zones appear to an observer the same color under at least two different illuminants, for example under at least two of the illuminants D65, D50 and A. Reference may be made beneficially in this respect to European patent application EP 1 212 961, the content of which is incorporated herein by reference.

Preferably, the spectral reflectances for at least some or even all of the above zones are within 25%, better still 20%, even better still 15%, even better still 10% and even better still 5% of those given in Table I, for at least one of the wavelengths indicated in the table and preferably all the wavelengths given therein.

The spectral reflectances are measured using a Minolta® spectrocolorimeter, of reference CM2002, in reflection mode, specular included, medium aperture.

The support 33 is, for example, a sheet of matt paper of 130 g/cm² for inkjet printing.

The contrast card 30 may be representative of skins with a lightness of between 50 and 60.

The various zones D6, D5, D7, D8 and C11 of the contrast card 30 may thus correspond to mean colors observed on a panel of individuals having a dark skin with a fair complexion for different regions of the face.

For example, the zone D6 may correspond to a mean of the colors observed on the forehead and the cheek bones, the color being measured on the forehead in zones V1 of the image of the face, as may be seen in FIG. 3, and on the cheekbones in zones V2. Zone D5 corresponds, for example, to the color of the cheekbones, measured in zone V2 of the image, zone D7, for example, to the color of the forehead in the zones V1, zone D8 may correspond to a mean of the color of the outer eye shadows measured in the zone V3, that of the middle eye shadows measured in the zone V4 and that measured around the lips in the zones V5. The zone C11 may correspond to the color measured on the inner eye shadow in the zone V6.

The contrast card 31 may be representative of skins whose lightness is between 40 and 50.

Thus, the various zones C9, C8, C10, C11 and B12 of the contrast card 31 may correspond to mean colors observed on a panel of individuals having a dark skin with a medium complexion for different regions of the face.

The zone C9 corresponds, for example, to the color measured on the forehead, in the zones V1 of the image averaged with the colored measured on the cheekbones, in the zones V2.

The zone C8 may correspond to the color measured on the cheekbones, in the zone V2 of the image. The zone C10 may correspond to the color measured on the forehead, in the zones V1.

The zone C11 corresponds, for example, to the average color between that of the outer eye shadows, measured in the zone V3, that of the middle eye shadows, measured in the zone V4, and that of the region around the lips, in the zone V5. The region B12 may correspond to the color of the inner eye shadows, measured in the region V6.

The contrast card 32 may be representative of skins whose lightness is between 30 and 40.

Thus, the various zones B11+, B11, B12, B12+ and XXX may correspond to mean colors observed on a panel of individuals having a dark skin with a dark complexion for different regions of the face.

The region B11+ may correspond to the mean of the color measured on the forehead in the zones V1 averaged with that measured on the cheekbones, in the zones V2.

The zone B11 may correspond to the color measured on the cheekbones, in the zone V2, the zone B12 to that measured on the forehead, in the zones V1, the zone B12+ to the mean color between that measured on the outer eye shadows in the zones V3, that measured in the middle eye shadows in the zones V4 and that measured around the lips in the zones V5. The zone XXX may correspond to the color of the inner eye shadows, measured in the zones V6.

A contrast card 32 was made, on which the following values could be measured, for each zone:

| | Zone | | | | |
|---|---|---|---|---|---|
| | B11+ | XXX | B12+ | B12 | B11 |
| L* | 36.7 | 29.63 | 32.98 | 35.66 | 38.43 |
| C* | 19.81 | 15.06 | 17.29 | 19.78 | 21.76 |
| h | 47.34° | 40.34° | 44.64° | 46.32° | 46.51° |

A contrast card 31 was made, on which the following values could be measured, for each zone:

| | Zone | | | | |
|---|---|---|---|---|---|
| | C9 | B12 | C11 | C10 | C8 |
| L* | 45.04 | 35.66 | 38.73 | 42.19 | 48.06 |
| C* | 25.18 | 19.78 | 21.94 | 24.18 | 25.97 |
| h | 53.27° | 46.32° | 50.18° | 51.94° | 53.09° |

A contrast card 30 was made, on which the following values could be measured, for each zone:

| | Zone | | | | |
|---|---|---|---|---|---|
| | D6 | C11 | D8 | D7 | D5 |
| L* | 54.08 | 38.73 | 47.94 | 51.79 | 57.61 |
| C* | 26.70 | 21.94 | 26.18 | 27.21 | 26.22 |
| h | 57.35° | 50.18° | 56.82° | 57.09° | 55.09° |

The measurements were taken using a Minolta® spectrocolorimeter, of reference CM2002, in reflection mode, specular included, medium aperture.

The spectral reflectances measured are those given in Table I, these values being given purely as non-limiting examples.

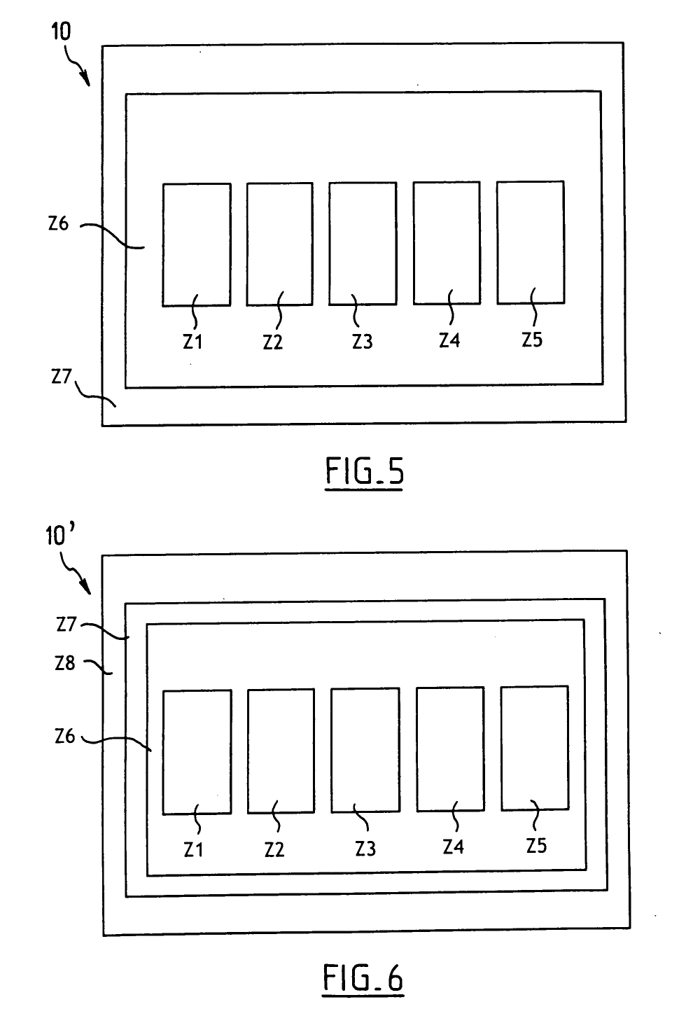

FIGS. 5 and 6 show other examples of contrast cards 10 and 10′ each comprising five colored zones Z1, Z2, Z3, Z4 and Z5, for example rectangular zones, arranged side by side at the centre of a sixth zone Z6 occupying, in the examples under consideration, a large portion of the background of the card.

The cards 10 and 10′ also comprise a white boder Z7 extending around the zone Z6 in the manner of a frame and the card 10′ differs from the card 10 only in the fact that it also comprises a black zone Z8 extending around the white zone Z7. This black zone Z8 may make it possible to evaluate, with the white zone Z7, the coverage of the composition in a manner similar to that allowed by the usual black/white contrast cards.

The various zones Z1 to Z7, and optionally Z8, are preferably made by printing inks chosen such that these zones appear to an observer the same color under at least two different illuminants, for example under at least two of the illuminants, D65, D50 and A.

The various zones Z1 to Z6 may correspond to mean colors observed on a panel of individuals having a dark skin, for example a skin belonging to a given type of black skin, for different regions of the face and the body. Skins with a lightness of between 45 and 50 (fair-tone dark skins), between 39 and 44 (medium-tone dark skins), and less than 39 (dark-tone dark skins) may be considered, for example, as being of the same type.

The zones Z1 to Z5 may correspond, for example, respectively, to the mean color measured on the forehead, the bags under the eyes, the space between the nose and the lips, and also on marks on the face, especially marks present on the cheeks, and the zone Z6 to the color measured on the body, for example the underside of the forearm.

In the example under consideration, the zones Z1, Z2 and Z3 of the card 10 correspond, respectively, to the color of the regions R1, R2 and R3 represented in FIG. 2.

A card 10 was prepared, on which the following values could be measured for each zone:

Zone

| | Z1 | Z2 | Z3 | Z4 | Z5 | Z6 |
|---|---|---|---|---|---|---|
| L* | 48.38 | 46.67 | 44.5 | 42.72 | 44.41 | 52.26 |
| a* | 7.99 | 6.78 | 6.76 | 4.12 | 6.57 | 9.11 |
| b* | 3.85 | 3.25 | 3.1 | 2.57 | 3.93 | 5.81 |

The measurements were taken using a Minolta® spectrocolorimeter, of reference CM3700d, in reflection mode, specular included, UV included, small aperture d/8.

For all the contrast cards, the values of L* and h are given to within 15%, or even 12.5%, better still 10%, or even 7.5%, or even 5%, and the values of C* are given to within 25%, better still 20%, even better still 15%, or even 10%, or even 5%.

Formulations

Compounds for preparing lightening compositions, which comprise at least one coloring agent, preferably combined with reflective particles, will now be described in the rest of the examples. The lightening compositions may also comprise fillers. The physiologically acceptable medium may comprise an aqueous phase or a fatty phase. The composition may also comprise at least one surfactant and/or at least one film-forming polymer.

Coloring Agents

Coloring agents that are suitable for the invention can produce, alone or as a mixture, a yellow or orange coloration. In other words, they have a significant reflectance in the range from 550 to 675 nm.

The coloring agent(s) may be present in the composition of foundation type, especially the base or surface composition of the product of foundation type according to the invention, in a content ranging from 0.5% to 30% by weight, especially ranging from 2% to 20% by weight and in particular from 5 to 18% by weight relative to the total weight of the composition under consideration.

The coloring agent(s) may be chosen from mineral or organic pigments, coloring polymers, water-soluble or liposoluble dyes, organic lacquers, metallic powders, and mixtures thereof. They may be chosen especially from those mentioned in the CTFA Cosmetic Ingredient Handbook, 3rd Edition Cosmetic and Fragrance Association., Inc., Washington D.C. (1982).

As nonlimiting illustrations of mineral coloring agents, mention may be made more particularly of yellow, red and brown metal oxides, for instance iron oxides.

As metallic powders, mention may be made of copper powder.

The pigments FDC Yellow No. 5 (disodium salt of tartrazine) are suitable especially as organic pigments.

As illustrations of organic lacquers that are suitable for the invention, mention may be made more particularly of FDC Yellow No. 5 and No. 6 A1 Lake.

The water-soluble dyes may be chosen, for example, from the brown dye identified by the name “caramel” according to the Color Index; the yellow dyes identified by the Color Index numbers 10316, 13015, 18690, 18820, 18965, 19140, 45430, 47005, 75100 and that known as Lactoflavin; the orange dyes identified by the Color Index numbers 14270, 15510, 15980, 15985, 16230, 20170, 40215; the red dyes identified by the Color Index numbers 14700, 14720, 14815, 15620, 16035, 16185, 16255, 16290, 17200, 18050, 18130, 18736, 24790, 27290, 45100, 45220, 45380, 45405, 45410, 45425, 45430, 75470, and mixtures thereof.

The liposoluble dyes may be chosen, for example, from the brown dye identified by the Color Index number 12010; the yellow dyes identified, respectively, by the Color Index numbers 12700, 21230, 47000, 75125, 75135; the orange dyes identified by the Color Index numbers 11920, 40800, 40820, 40825, 40850, 45396, 75120, 75130 and capasanthine and the red dye identified by number 12150, and mixtures thereof.

The dyeing polymer is a polymer comprising at least one organic dye group. The dye group may be grafted, especially via covalent bonding, onto the chain of the polymer. The dye polymer generally contains less than 10% by weight of dyestuff relative to the total weight of the polymer.

This dye polymer may be of any chemical nature, especially polyester, polyamide, polyurethane, polyacrylic, poly(meth)acrylic, polycarbonate, of natural origin, for instance cellulose or chitosan polymers, or a mixture thereof, and in particular a polyester or polyurethane.

In particular, the dye polymer may be a copolymer based on at least two different monomers, at least one of which is an organic dye monomer.

Such dye polymers are described especially in patents or patent applications U.S. Pat. No. 5,032,670, U.S. Pat. No. 4,999,418, U.S. Pat. No. 5,106,942, U.S. Pat. No. 5,030,708, U.S. Pat. No. 5,102,980, U.S. Pat. No. 5,043,376, U.S. Pat. No. 5,104,913, U.S. Pat. No. 5,281,659, U.S. Pat. No. 5,194,463, U.S. Pat. No. 4,804,719, WO 92/07913 and EP-A-747036.

As illustrations of monomers for known dye polymers, mention may be made especially of anthraquinones, methines, bis-methines, azamethines, arylidenes, 3H-dibenzo[7,i-j]isoquinolines, 2,5-diarylaminoterephthalic acids and esters thereof, phthaloylphenothiazines, phthaloylphenoxazines, phthaloylacridone, anthrapyrimidines, anthrapyrazoles, phthalocyanins, quinophthalones, indophenols, perinones, nitroarylamines, benzodifuran, 2H-1-benzopyran-2-one, quinophthalones, perylenes, quinacridones, triphenodioxazines, fluoridines, 4-amino-1,8-naphthalimides, thioxanthrones, benzanthrones, indanthrones, indigo, thioindigo, xanthene, acridine, azine and oxazine.

Needless to say, a person skilled in the art is capable, via his general knowledge, of selecting the monomers to adjust the desired color effect according to the invention.

The color agent(s) and especially the pigments used in the context of the present invention may be used either in their crude form or in a pretreated form, especially pretreated at the surface thereof. The aim of this treatment is generally to increase the stability of the color and to facilitate their incorporation into cosmetic formulations. In particular, coloring agents treated in order to make them hydrophobic will be more readily dispersible in an oily phase.

An illustration of these surface treatments that may be mentioned especially is the treatment consisting in treating the pigment with a hydrophobic and oil-repellant agent of perfluoroalkyl phosphate derivative type, as described in EP 1 086 683.

Similarly, it may be useful to treat the coloring agents, and especially the pigments, with a material that makes them compatible with the oily phases and especially the silicone phases used in cosmetic formulations. Pigments of this type are described especially in patent U.S. Pat. No. 5,143,722.

According to one particular embodiment, the coloring agents used according to the invention are of yellow, orange, brown or red color.

As illustrations of coloring agents that are more particularly suitable for the invention, mention may be made especially of brown iron oxide and yellow iron oxide, coated with perfluoroalkyl phosphate, and titanium oxide treated with alumina, coated with perfluoroalkyl phosphate, or titanium oxide coated with perfluoroalkyl phosphate, for instance, in particular, the pigmentary pastes sold under the trade names Yellow Iron Oxide Covafluor, PF5 Yellow 601 (yellow) and PF5 Red R516L (red), PF 5 Black BL100 by the company Daito, under the trade names FA50DRF, FA50DYF, FA65DF and FA65 DBF by the company Kobo, ultramarine blue coated with perfluoroalkyl phosphate, under the trade name PF 5 Ultramarine No. 801 by the company Daito, the disodium salts of tartrazine and the aluminium lakes of Allura red on alumina sold by the company Noveon under the names FDC Yellow No. 6, A1 Lake and FDC Yellow No. 5 A1 Lake, and mixtures thereof.

Reflective Particles

For the purposes of the present invention, the term “reflective particles” denotes particles whose size, structure, especially the thickness of the layer(s) of which they are composed and their physical and chemical natures, and surface state allow them to reflect incident light. This reflection may, where appropriate, have an intensity sufficient to create at the surface of the composition of foundation type according to the invention, when this composition is applied to the support to be made up, highlight points that are visible to the naked eye, i.e more luminous points which contrast with their environment and appear to shine.

The reflective particles are also selected so as not to significantly impair the coloring effect generated by the coloring agents combined therewith and more particularly so as to optimize this effect in terms of color rendition. They may more particularly have a yellow, pink, red, bronze, orange, brown, gold and/or coppery color or glint.

The reflective particles may be present in the composition according to the invention and the base or surface composition of the product according to the invention in a content ranging from 0.5% to 60% relative to the total weight of the composition, especially from 1% to 30% by weight, in particular from 2% to 20% by weight or even from 3% to 10% by weight.

These particles may have varied forms. These particles may especially be in the form of platelets or in globular form, in particular in spherical form.

Whatever their form, the reflective particles may have a multilayer or non-multilayer structure and, in the case of a multilayer structure, for example at least one layer of uniform thickness, especially of a reflective material.

When the reflective particles do not have a multilayer structure, they may be composed, for example, of metal oxides, for example titanium oxide or iron oxide obtained synthetically.

When the reflective particles have a multilayer structure, they may, for example, comprise a natural or synthetic substrate, especially a synthetic substrate at least partially coated with at least one layer of a reflective material especially of at least one metal or metallic compound. The substrate may be monomaterial or multimaterial, and organic and/or mineral.

More particularly, it may be chosen from glasses, ceramics, graphite, metal oxides, aluminas, silicas, silicates, especially aluminosilicates and borosilicates, synthetic mica and mixtures thereof, this list not being limiting.

The reflective material may comprise a layer of metal or of a metallic compound.

Glass particles coated with a metallic layer are described especially in documents JP-A-09 188 830, JP-A-10 158 450, JP-A-10 158 541, JP-A-07 258 460 and JP-A-05 017 710.

Again as examples of reflective particles comprising a mineral substrate coated with a layer of metal, mention may also be made of particles comprising a borosilicate substrate coated with silver, also known as “white nacres”.

Particles containing a glass substrate coated with silver, in the form of platelets, are sold under the name Microglass Metashine REFSX 2025 PS by the company Toyal. Particles containing a glass substrate coated with a nickel/chromium/molybdenum alloy are sold under the name Crystal Star GF 550, GF 2525 by this same company.

Irrespective of their form, the reflective particles may also be chosen from particles containing a synthetic substrate coated at least partially with at least one layer of at least one metallic compound, especially a metal oxide chosen, for example, from titanium oxide, especially TiO₂, iron oxide, especially Fe₂O₃, tin oxide, chromium oxide, barium sulphate and the following compounds: MgF₂, CrF₃, ZnS, ZnSe, SiO₂, Al₂O₃, MgO, Y₂O₃, SeO₃, SiO, HfO₂, ZrO₂, CeO₂, Nb₂O₅, Ta₂O₅, MoS₂ and mixtures or alloys thereof.

As examples of such particles, mention may be made, for example, of particles comprising a synthetic mica substrate coated with titanium dioxide, or glass particles coated either with brown iron oxide, titanium oxide, tin oxide or with one of the mixtures thereof, such as those sold under the brand name Reflecks® by the company Engelhard.

The reflective particles may or may not be goniochromatic and/or may or may not be interference particles. For the purposes of the invention, they comprise nacres and goniochromatic coloring agents.

The term “nacres” should be understood as meaning colored particles of any form, which may or may not be iridescent, produced especially by certain molluscs in their shell or else synthesized, and which have a color effect by optical interference.

The nacres may be chosen from nacreous pigments such as titanium mica coated with an iron oxide, mica coated with bismuth oxychloride, titanium mica coated with chromium oxide, titanium mica coated with an organic dye, especially of the abovementioned type, and also nacreous pigments based on bismuth oxychloride. They may also be mica particles at the surface of which are superposed at least two successive layers of metal oxides and/or of organic dyestuffs.

The nacres may more particularly have a yellow, pink, red, bronze, orange, brown, gold and/or coppery color or glint.

As illustrations of nacres that may be used in the context of the present invention, mention may be made especially of the gold-colored nacres sold especially by the company Engelhard under the name Brillant Gold 212G (Timica), Gold 222C (Cloisonne), Sparkle Gold (Timica), Gold 4504 (Chromalite) and Monarch Gold 233×(Cloisonne); the bronze nacres sold especially by the company Merck under the name Bronze Fine (17384) (Colorona) and Bronze (17353) (Colorona) and by the company Engelhard under the name Super Bronze (Cloisonne); the orange nacres sold especially by the company Engelhard under the name Orange 363C (Cloisonne) and Orange MCR 101 (Cosmica) and by the company Merck under the name Passion Orange (Colorona) and Matte Orange (17449) (Microna); the brown nacres sold especially by the company Engelhard under the name Nu-antique Copper 340XB (Cloisonne) and Brown CL4509 (Chromalite); the nacres with a copper glint sold especially by the company Engelhard under the name Copper 340A (Timica); the nacres with a red glint sold especially by the company Merck under the name Sienna Fine (17386) (Colorona); the nacres with a yellow glint sold especially by the company Engelhard under the name Yellow (4502) (Chromalite); the red nacres with a golden glint sold especially by the company Engelhard under the name Sunstone G012 (Gemtone); the pink nacres sold especially by the company Engelhard under the name Tan Opale G005 (Gemtone); the black nacres with a golden glint sold especially by the company Engelhard under the name Nu-antique Bronze 240 AB (Timica), the blue nacres sold especially by the company Merck under the name Matte Blue (17433) (Microna), the white nacres with a silvery glint sold especially by the company Merck under the name Xirona Silver, and the golden green pink-orange nacres sold especially by the company Merck under the name Indian Summer (Xirona), and mixtures thereof.

It may also be envisaged to use a goniochromatic coloring agent as reflective particles, with the proviso that this agent satisfies the hue effect stipulation required according to the invention and does not otherwise disrupt the visual perception of the composition in terms of the color effect. This goniochromatic coloring agent may be chosen especially from multilayer interference structures.

Fillers

As mentioned previously, the presence of reflective particles in a proportion sufficient to ensure a filler function makes it possible to significantly reduce the amount, or even to avoid the presence, of conventional filler(s) of white particle type.

In the present case, the compositions of foundation type in accordance with the invention may contain less than 5% or even 3% by weight of white particles and especially of titanium oxide, or may even be free of titanium oxide.

Needless to say, it nevertheless remains possible to combine the reflective particles according to the invention with one or more other conventional fillers, with the proviso that these conventional fillers are used in an amount such that they do not affect the aesthetic effect desired by the compositions of foundation type claimed, i.e they do not excessively impart a greyish appearance to the made-up skin when this skin is coated with a composition in accordance with the invention. A person skilled in the art is capable, by means of his knowledge, of making this adjustment.

These fillers may be mineral or organic and of any form: platelet, spherical or oblong, irrespective of the crystallographic form (for example leaflet, cubic, hexagonal, orthorhombic, etc.). Mention may be made of talc, mica, silica, kaolin, polyamide powder (Nylon) (Orgasol® from Atochem), poly-α-alanine powder and polyethylene powder, tetrafluoroethylene polymer powders (Teflon), lauroyllysine, starch, boron nitride, hollow polymer microspheres such as those made of polyvinylidene chloride/acrylonitrile, for instance Expancel® (Nobel Industrie), made of acrylic acid copolymers (Polytrap® from the company Dow Corning) or made of polymethyl methacrylate (Covabead from Wackherr), silicone resin microbeads (for example Tospearls® from Toshiba), polyorganosiloxane elastomer particles, precipitated calcium carbonate, magnesium carbonate, magnesium hydrocarbonate, hydroxyapatite, hollow silica microspheres (Silica Beads® from Maprecos), glass or ceramic microcapsules, metal soaps derived from organic carboxylic acids containing from 8 to 22 carbon atoms and preferably from 12 to 18 carbon atoms, for example zinc, magnesium or lithium stearate, zinc laurate or magnesium myristate, and mixtures thereof.

It may be advantageous to favour the choice of transparent complementary fillers, for instance filmed silica.

This or these filler(s) may be present in a proportion of from 0.1% to 20% by weight, preferably 2% to 15% by weight and better still from 2% to 10% by weight relative to the total weight of the composition of foundation type, especially of the base or surface compositions of the product of foundation type.

Physiologically Acceptable Medium

The term “physiologically acceptable medium” denotes a non-toxic medium that may be applied to human skin. The physiologically acceptable medium is generally suited to the nature of the skin onto which the composition of foundation type is to be applied and also to the form in which the composition is intended to be packaged, especially fluid at room temperature and at atmospheric pressure.

As mentioned previously, the compositions of foundation type according to the invention, especially the base and/or surface compositions of the product of foundation type according to the invention, may be formulated in a fluid or solid form of free, compact or cast powder type.

They may especially be, independently of each other, in an anhydrous form or in the form of a gel, of direct, inverse or multiple emulsion combining at least one aqueous phase and at least one fatty phase.

Aqueous Phase

The composition of foundation type according to the invention, especially the base composition and/or the surface composition of the product of foundation type according to the invention, may comprise at least one aqueous medium, constituting an aqueous phase, which may form the continuous phase of the composition of foundation type under consideration.

The aqueous phase may consist essentially of water.

It may also comprise a mixture of water and of water-miscible organic solvent (miscible in water to greater than 50% by weight at 25° C.), for instance lower monoalcohols containing from 1 to 5 carbon atoms, such as ethanol, isopropanol, glycols containing from 2 to 8 carbon atoms, such as propylene glycol, ethylene glycol, 1,3-butylene glycol, dipropylene glycol, C₃-C₄ ketones and C₂-C₄ aldehydes.

The aqueous phase (water and optionally the water-miscible organic solvent) may be present in a content ranging from 1% to 95% by weight, especially ranging from 3% to 80% by weight, and in particular ranging from 5% to 60%, by weight relative to the total weight of the composition under consideration.

Such a medium may also comprise a volatile oil as defined below.

Fatty Phase

The composition of foundation type according to the invention, especially the base composition and/or the surface composition of the product of foundation type according to the invention, may comprise a fatty phase and especially at least one fatty substance that is liquid at room temperature (25° C.) and/or a fatty substance that is solid at room temperature, such as waxes, pasty fatty substances and gums, and mixtures thereof. The fatty phase may also contain lipophilic organic solvents.

The composition of foundation type may contain, for example, a continuous fatty phase, which may contain less than 5% water, especially less than 1% water, relative to its total weight, and may in particular be in anhydrous form.

The fatty phase of the composition according to the invention may especially comprise, as liquid fatty substance, at least one volatile or non-volatile oil or a mixture thereof.

For the purposes of the invention, the term “volatile oil” means any oil capable of evaporating on contact with the skin in less than one hour, at room temperature and atmospheric pressure. The volatile oils of the invention are volatile cosmetic oils, which are liquid at room temperature, having a non-zero vapour pressure, at room temperature and atmospheric pressure, ranging in particular from 0.01 to 300 mmHg (1.33 Pa to 40 000 Pa) and preferably greater than 0.3 mmHg (30 Pa).

The term “non-volatile oil” means an oil that remains on the skin at room temperature and atmospheric pressure for at least several hours and that especially has a vapour pressure of less than 0.01 mmHg (1.33 Pa).

These volatile or non-volatile oils may be hydrocarbon-based oils, silicone oils or mixtures thereof. The term “hydrocarbon-based oil” means an oil mainly containing hydrogen and carbon atoms and possibly oxygen, nitrogen, sulphur and phosphorus atoms.

The volatile hydrocarbon-based oils may be chosen from hydrocarbon-based oils containing from 8 to 16 carbon atoms, and especially branched C₈-C₁₆ alkanes, for instance C₈-C₁₆ isoalkanes of petroleum origin (also known as isoparaffins), for instance isododecane (also known as 2,2,4,4,6-pentamethylheptane), isodecane, isohexadecane, and for example the oils sold under the trade names Isopars® or Pemmetyls®, branched C₈-C₁₆ esters such isohexyl neopentanoate, and mixtures thereof. Other volatile hydrocarbon-based oils, for instance petroleum distillates, especially those sold under the name Shell Solt® by the company Shell, may also be used.

Volatile oils that may also be used include volatile silicones, for instance volatile linear or cyclic silicone oils, especially those with a viscosity≦8 centistokes (8×10⁻⁶ m²/s) and especially containing from 2 to 7 silicon atoms, these silicones optionally comprising alkyl or alkoxy groups containing from 1 to 10 carbon atoms. As volatile silicone oils that may be used in the invention, mention may be made especially of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

The volatile oil may be present in a composition according to the invention in a content ranging from 0.1% to 98% by weight, especially from 1% to 65% by weight, and in particular from 2% to 50% by weight, relative to the total weight of the composition.

The non-volatile oils may be chosen especially from non-volatile fluoro and/or silicone hydrocarbon-based oils.

Non-volatile hydrocarbon-based oils that may especially be mentioned include:
hydrocarbon-based oils of animal origin,
hydrocarbon-based oils of plant origin, such as triglycerides consisting of fatty acid esters of glycerol, the fatty acids of which may have varied chain lengths from C₄ to C₂₄, these chains possibly being linear or branched, and saturated or unsaturated;
these oils are especially wheatgerm oil, sunflower oil, grapeseed oil, sesame seed oil, maize oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppyseed oil, pumpkin oil, sesame seed oil, marrow oil, rapeseed oil, blackcurrant oil, evening primrose oil, millet oil, barley oil, quinoa oil, rye oil, safflower oil, candlenut oil, passionflower oil or musk rose oil; shea butter; or caprylic/capric acid triglycerides, for instance those sold by the company Stearineries Dubois or those sold under the names Miglyol 810, 812 and 818® by the company Dynamit Nobel,
synthetic ethers containing from 10 to 40 carbon atoms,
linear or branched hydrocarbons of mineral or synthetic origin, such as petroleum jelly, polydecenes, hydrogenated polyisobutene such as parleam, and squalane, and mixtures thereof,
synthetic esters, for instance oils of formula R₁COOR₂ in which R₁ represents a linear or branched fatty acid residue containing from 1 to 40 carbon atoms and R₂ represents a hydrocarbon-based chain, which is especially branched, containing from 1 to 40 carbon atoms, on condition that R₁+R₂>10, for instance purcellin oil (cetostearyl octanoate), isopropyl myristate, isopropyl palmitate, C₁₂ to C₁₅ alkyl benzoates, hexyl laurate, diisopropyl adipate, isononyl isononanoate, 2-ethylhexyl palmitate, isostearyl isostearate, alcohol or polyalcohol heptanoates, octanoates, decanoates or ricinoleates, for instance propylene glycol dioctanoate; hydroxylated esters, for instance isostearyl lactate or diisostearyl malate; polyol esters and pentaerythritol esters,
fatty alcohols that are liquid at room temperature with a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpentadecanol,
higher fatty acids such as oleic acid, linoleic acid or linolenic acid, and mixtures thereof.

The non-volatile silicone oils that may be used in the composition according to the invention may be non-volatile polydimethylsiloxanes (PDMS), polydimethylsiloxanes comprising alkyl or alkoxy groups, which are pendant and/or at the end of a silicone chain, these groups each containg from 2 to 24 carbon atoms, phenylsilicones, for instance phenyl trimethicones, phenyl dimethicones, phenyl trimethylsiloxy diphenylsiloxanes, diphenyl dimethicones, diphenyl methyldiphenyl trisiloxanes and 2-phenylethyl trimethylsiloxysilicates.

The non-volatile oils may be present in a composition according to the invention in a content ranging from 0.01% to 90% by weight, especially from 0.1% to 85% by weight and in particular from 1% to 70% by weight relative to the total weight of the composition.

More generally, the liquid fatty substance may be present in a proportion of from 0.01% to 90% by weight and especially from 0.1% to 85% by weight relative to the weight of the fatty phase.

As regards the fatty substance that is solid at room temperature and atmospheric pressure, it may be chosen from waxes, pasty fatty substances and gums, and mixtures thereof. This solid fatty substance may be present in a proportion of from 0.01% to 50%, especially from 0.1% to 40% and in particular from 0.2% to 30% by weight relative to the total weight of the fatty phase.

Thus, a composition according to the invention may comprise at least one fatty substance that is pasty at room temperature.

For the purposes of the invention, the term “pasty fatty substance” means fatty substances with a melting point ranging from 20 to 55° C. and preferably 25 to 45° C., and/or a viscosity at 40° C. ranging from 0.1 to 40 Pa.s (1 to 400 poises) and preferably 0.5 to 25 Pa.s, measured using a Contraves TV or Rheomat 80 viscometer, equipped with a spindle rotating at 60 Hz. A person skilled in the art can select the spindle for measuring the viscosity from the spindles MS-r3 and MS-r4, on the basis of his general knowledge, so as to be able to perform the measurement on the pasty compound tested.

Preferably, these fatty substances are hydrocarbon-based compounds optionally of polymeric type; they may also be chosen from silicone compounds; they may also be in the form of a mixture of hydrocarbon-based and/or silicone compounds. In the case of a mixture of different pasty fatty substances, the hydrocarbon-based pasty compounds (mainly containing carbon and hydrogen atoms and optionally ester groups) are preferably used, in majority proportion.

Among the pasty compounds that may be used in the composition of foundation type according to the invention, mention may be made of lanolins and lanolin derivatives, for instance acetylated lanolins, oxypropylenated lanolins or isopropyl lanolate, with a viscosity of from 18 to 21 Pa.s and preferably 19 to 20.5 Pa.s, and/or a melting point of from 30 to 55° C., and mixtures thereof. Esters of fatty acids or of fatty alcohols, especially those containing 20 to 65 carbon atoms (melting point from about 20 to 35° C. and/or viscosity at 40° C. ranging from 0.1 to 40 Pa.s) may also be used, for instance triisostearyl citrate or cetyl citrate; arachidyl propionate; polyvinyl laurate; cholester esters, for instance triglycerides of plant origin such as hydrogenated plant oils, viscous polyesters, for instance poly(12-hydroxystearic acid), and mixtures thereof. Triglycerides of plant origin that may be used include hydrogenated castor oil derivatives, such as “Thixinr” from Rheox.

Mention may also be made of silicone pasty fatty substances such as polydimethylsiloxanes (PDMS) of high molecular weight and in particular those with pendant chains of the alkyl or alkoxy type containing from 8 to 24 carbon atoms, and a melting point of 20-55° C., for instance stearyl dimethicones, especially those sold by the company Dow Corning under the trade names DC2503® and DC25514®, and mixtures thereof.

The pasty fatty substance may be present in a composition according to the invention in a content ranging from 0.01% to 50% by weight, preferably ranging from 0.1% to 45% by weight and better still ranging from 0.2% to 30% by weight relative to the total weight of the said composition.

The composition of foundation type according to the invention, especially the base composition and/or the surface composition of the product of foundation type according to the invention, may also comprise a wax. The wax may be solid at room temperature (25° C.), with a reversible solid/liquid change of state, having a melting point of greater than 30° C. which may be up to 200° C., a hardness of greater than 0.5 MPa and having an anisotropic crystalline organization in the solid state. It may be a hydrocarbon-based wax, a fluoro wax and/or a silicone wax and may be of animal, plant, mineral or synthetic origin. It may be chosen, for example, from beeswax, carnauba wax, candelilla wax, paraffin waxes, hydrogenated castor oil, silicone waxes or microcrystalline waxes, and mixtures thereof.

In particular, the wax may be present in the form of a wax-in-water emulsion.

The wax may be present in a composition according to the invention in a content ranging from 0.01% to 50% by weight, in particular from 0.1% to 30% by weight and especially from 0.2% to 20% by weight relative to the total weight of the composition.

Surfactants

The composition of foundation type according to the invention, especially the base composition and/or the surface composition of the product of foundation type according to the invention, may also contain emulsifying surfactants present especially in a proportion ranging from 0.1 to 30% by weight and better still from 5% to 15% by weight relative to the total weight of the composition.

These surfactants may be chosen from anionic and nonionic surfactants. Reference may be made to the document “Encyclopedia of Chemical Technology, Kirk-Othmer”, volume 22, pp. 333-432, 3rd edition, 1979, Wiley, for the definition of the properties and functions (emulsifying) of surfactants, in particular pp. 347-377 of this reference, for the anionic and nonionic surfactants.

The surfactants preferably used in the composition of foundation type according to the invention are chosen from:
nonionic surfactants: fatty acids, fatty alcohols, polyethoxylated or polyglycerolated fatty alcohols such as polyethoxylated stearyl or cetylstearyl alcohol, fatty acid esters of sucrose, alkylglucose esters, in particular polyoxyethylenated C₁-C₆ alkyl glucose fatty esters, and mixtures thereof,
anionic surfactants: C₁₆-C₃₀ fatty acids neutralized with amines, aqueous ammonia or alkaline salts, and mixtures thereof.

Surfactants that allow oil-in-water or wax-in-water emulsions to be obtained are preferably used.

Film-Forming Polymer

The composition of foundation type according to the invention, especially the base composition and/or the surface composition of the product of foundation type according to the invention, may also comprise at least one film-forming polymer.

In the present patent application, the term “film-forming polymer” means a polymer capable of forming, by itself or in the presence of an auxiliary film-forming agent, a continuous film that adheres to the skin.

A film-forming polymer capable of forming a hydrophobic film, i.e a polymer whose film has a solubility in water at 25° C. of less than 1% by weight, is preferably used.

The film-forming polymer may especially be at least one polymer chosen from the group comprising:
water-soluble film-forming polymers,
aqueous dispersions of water-dispersible film-forming polymer particles, also known as “latices”; in this case, the composition of foundation type should comprise an aqueous phase,
liposoluble film-forming polymers,
lipodispersible film-forming polymers in the form of non-aqueous dispersions of polymer particles, preferably dispersions of polymer particles, where appropriate surface-stabilized with at least one stabilizer, in one or more silicone and/or hydrocarbon-based oils; these non-aqueous dispersions are also known as “NADs”.

The composition of foundation type may in parallel comprise a mixture of these polymers.

The film-forming polymer may be present in a composition according to the invention in a solids content ranging from 0.01% to 20% by weight and especially from 0.5% to 10% by weight relative to the total weight of the composition.

Among the film-forming polymers that may be used according to the invention, mention may be made of synthetic polymers, of free-radical type or of polycondensate type, polymers of natural origin, and mixtures thereof.

The term “free-radical film-forming polymer” means a polymer obtained by polymerization of unsaturated monomers, especially ethylenically unsaturated monomers, each monomer being capable of homopolymerizing (unlike polycondensates).

The film-forming polymers of free-radical type may especially be vinyl polymers or copolymers, especially acrylic polymers.

The vinyl film-forming polymers may result from the polymerization of ethylenically unsaturated monomers containing at least one acid group and/or esters of the acidic monomers and/or amides of these acidic monomers.

α,β-Ethylenic unsaturated carboxylic acids such as acrylic acid, methacrylic acid, crotonic acid, maleic acid or itaconic acid may be used as monomer bearing an acid group. (Meth)acrylic acid and crotonic acid are preferably used, and more preferably (meth)acrylic acid.

The esters of acidic monomers are advantageously chosen from (meth)acrylic acid esters (also known as meth)acrylates), especially alkyl (meth)acrylates, in particular of a C₁-C₃₀ alkyl and preferably a C₁-C₂₀ alkyl, aryl (meth)acrylates, in particular of a C₆-C₁₀ aryl, and hydroxyalkyl (meth)acrylates, in particular of a C₂-C₆ hydroxyalkyl.

Among the alkyl (meth)acrylates that may be mentioned are methyl methacrylate, ethyl methacrylate, butyl methacrylate, isobutyl methacrylate, 2-ethylhexyl methacrylate, lauryl methacrylate and cyclohexyl methacrylate.

Among the hydroxyalkyl (meth)acrylates that may be mentioned are hydroxyethyl acrylate, 2-hydroxypropyl acrylate, hydroxyethyl methacrylate and 2-hydroxypropyl methacrylate.

Among the aryl (meth)acrylates that may be mentioned are benzyl acrylate and phenyl acrylate.

The (meth)acrylic acid esters that are particularly preferred are alkyl (meth)acrylates.

According to the present invention, the alkyl group of the esters may be either fluorinated or perfluoriated, i.e some or all of the hydrogen atoms of the alkyl group are replaced with fluorine atoms.

Examples of amides of the acidic monomers that may be mentioned include (meth)acrylamides, and especially N-alkyl(meth)acrylamides, in particular of a C₂-C₁₂ alkyl. Among the N-alkyl (meth)acrylamides that may be mentioned are N-ethylacrylamide, N-t-butylacrylamide, N-t-octylacrylamide and N-undecylacrylamide.

The vinyl film-forming polymers may also result from the homopolymerization or copolymerization of monomers chosen from vinyl esters and styrene monomers. In particular, these monomers may be polymerized with acidic monomers and/or esters thereof and/or amides thereof, such as those mentioned previously.

Examples of vinyl esters that may be mentioned include vinyl acetate, vinyl neodecanoate, vinyl pivalate, vinyl benzoate and vinyl t-butylbenzoate.

Styrene monomers that may be mentioned include styrene and α-methylstyrene.

It is possible to use any monomer known to those skilled in the art falling within the categories of acrylic and vinyl monomers (including monomers modified with a silicone chain).

Among the film-forming polycondensates that may be mentioned are polyurethanes, polyesters, polyesteramides, polyamides, epoxyester resins and polyureas.

The polyurethanes may be chosen from anionic, cationic, nonionic and amphoteric polyurethanes, polyurethane-acrylics, polyurethanes-polyvinylpyrrolidones, polyester-polyurethanes, polyether-polyurethanes, polyureas and polyurea-polyurethanes, and mixtures thereof.

The polymers of natural origin, optionally modified, may be chosen from shellac resin, sandarac gum, dammar resins, elemi gums, copal resins and cellulose-based polymers, and mixtures thereof.

In a first embodiment of the composition of foundation type according to the invention, the film-forming polymer may be present in the form of particles in aqueous dispersion, which are generally known as latices or pseudolatices. The techniques for preparing these dispersions are well known to those skilled in the art.

Aqueous dispersions of film-forming polymers that may be used include the acrylic dispersions sold under the names Neocryl XK-90®, Neocryl A-1070@, Neocryl A-1090@, Neocryl BT-62®, Neocryl A-1079®, Neocryl A-523@ by the company Avecia-Neoresins, Dow Latex 4320 by the company Dow Chemical, Daitosol 5000 AD® by the company Daito Kasey Kogyo; or alternatively the aqueous polyurethane dispersions sold under the names Neorez R-981®, Neorez R-974® by the company Avecia-Neoresins, Avalure UR-405®, Avalure UR-410®, Avalure UR-425®, Avalure UR-450®, Sancure 875®, Sancure 861®, Sancure 878®, Sancure 2060® by the company Goodrich, Impranil 85® by the company Bayer and Aquamere H-1511 by the company Hydromer.

Aqueous dispersions of film-forming polymers that may also be used include the polymer dispersions resulting from the free-radical polymerization of one or more free-radical monomers within and/or partially at the surface of preexisting particles of at least one polymer chosen from the group consisting of polyurethanes, polyureas, polyesters, polyesteramides and/or alkyds. These polymers are generally known as hybrid polymers.

In a second embodiment of the composition of foundation type according to the invention, the film-forming polymer may be a water-soluble polymer and is thus present in the aqueous phase of the composition in dissolved form. Examples of water-soluble film-forming polymers that may be mentioned include:
proteins, for instance proteins of plant origin such as wheat or soybean proteins; proteins of animal origin such as keratins, for example keratin hydrolysates and sulphonic keratins;
anionic, cationic, amphoteric or nonionic chitin or chitosan polymers;
cellulose polymers such as hydroxyethylcellulose, hydroxypropylcellulose, methylcellulose, ethylhydroxyethylcellulose, carboxymethylcellulose and quaternized cellulose derivatives;
acrylic polymers or copolymers, such as polyacrylates or polymethacrylates;
vinyl polymers, for instance polyvinylpyrrolidones, copolymers of methyl vinyl ether and of maleic anhydride, the copolymer of vinyl acetate and of crotonic acid, copolymers of vinylpyrrolidone and of vinyl acetate; copolymers of vinylpyrrolidone and of vinylcaprolactam; polyvinyl alcohol;
polymers of natural origin, optionally modified, such as:
gum arabic, guar gum, xanthan derivatives and karaya gum;
alginates and carrageenans;
glycosaminoglycans, and hyaluronic acid and its derivatives;
shellac resin, sandarac gum, dammar resins, elemi gums and copal resins;
deoxyribonucleic acid;
mucopolysaccharides such as hyaluronic acid, chondroitin sulphates, and mixtures thereof.

According to another embodiment variant of the composition of foundation type according to the invention, the film-forming polymer may be present in a liquid fatty phase comprising organic oils or solvents such as those described above. For the purposes of the invention, the term “liquid fatty phase” means a fatty phase that is liquid at room temperature (25° C.) and atmospheric pressure (760 mm Hg, i.e 105 Pa), composed of one or more fatty substances that are liquid at room temperature, also known as oils, which are generally mutually compatible.

Preferably, the liquid fatty phase comprises a volatile oil, optionally mixed with a non-volatile oil, the oils possibly being chosen from the oils mentioned above.

In a third embodiment of the composition of foundation type according to the invention, the film-forming polymer may be in the form of surface-stabilized particles dispersed in the liquid fatty phase.

The dispersion of surface-stabilized polymer particles may be manufactured as described in document EP-A-749 747.

The polymer particles are surface-stabilized by means of a stabilizer, which may be a block polymer, a grafted polymer and/or a random polymer, alone or as a mixture.

Dispersions of film-forming polymer in the liquid fatty phase, in the presence of stabilizers, are described especially in documents EP-A-749 746, EP-A-923 928 and EP-A-930 060, the content of which is incorporated by reference into the present patent application.

The size of the polymer particles in dispersion either in the aqueous phase or in the liquid fatty phase may range from 5 nm to 600 nm and preferably from 20 nm to 300 nm.

In a fourth embodiment of the composition of foundation type according to the invention, the film-forming polymer may be dissolved in the liquid fatty phase, in which case the film-forming polymer is said to be a liposoluble polymer.

Examples of liposoluble polymers that may be mentioned include copolymers of a vinyl ester (the vinyl group being directly attached to the oxygen atom of the ester group and the vinyl ester having a saturated, linear or branched hydrocarbon-based radical of 1 to 19 carbon atoms, linked to the carbonyl of the ester group) and of at least one other monomer, which may be a vinyl ester (other than the vinyl ester already present), an α-olefin (containing from 8 to 28 carbon atoms), an alkyl vinyl ether (the alkyl group of which contains from 2 to 18 carbon atoms) or an allylic or methallylic ester (containing a saturated, linear or branched hydrocarbon-based radical of 1 to 19 carbon atoms, linked to the carbonyl of the ester group).

These copolymers may be crosslinked using crosslinking agents, which have the aim [lacuna], which may be either of the vinyl type or of the allylic or methallylic type, such as tetraallyloxyethane, divinylbenzene, divinyl octanedioate, divinyl dodecanedioate, and divinyl octadecanedioate.

Examples of these copolymers that may be mentioned include the following copolymers: vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/-1 octadecene, vinyl acetate/1-dodecene, vinyl stearate/ethyl vinyl ether, vinyl propionate/cetyl vinyle ether, vinyl stearate/allyl acetate, vinyl 2,2-dimethyloctanoate/vinyl laurate, allyl 2,2-dimethyl-pentanoate/vinyl laurate, vinyl dimethylpropionate/vinyl stearate, allyl dimethylpropionate/vinyl stearate, vinyl propionate/vinyl stearate, crosslinked with 0.2% divinylbenzene, vinyl dimethylpropionate/vinyl laurate, crosslinked with 0.2% divinylbenzene, vinyl acetate/octadecyl vinyl ether, crosslinked with 0.2% tetraallyloxyethane, vinyl acetate/allyl stearate, crosslinked with 0.2% divinylbenzene, vinyl acetate/1-octadecene crosslinked with 0.2% divinylbenzene, and allyl propionate/allyl stearate, crosslinked with 0.2% divinylbenzene.

Liposoluble film-forming polymers that may also be mentioned include liposoluble homopolymers, and in particular those resulting from the homopolymerization of vinyl esters containing from 9 to 22 carbon atoms or of alkyl acrylates or methacrylates, the alkyl radicals containing from 10 to 20 carbon atoms.

Such liposoluble homopolymers may be chosen from polyvinyl stearate, polyvinyl stearate crosslinked with divinylbenzene, with diallyl ether or with diallyl phthalate, polystearyl (meth)acrylate, polyvinyl laurate and polylauryl (meth)acrylate, these poly(meth)acrylates possibly being crosslinked with ethylene glycol dimethacrylate or tetraethylene glycol dimethacrylate.

The liposoluble copolymers and homopolymers defined above are known and described especially in patent application FR-A-2 232 303; they may have a weight-average molecular weight ranging from 2000 to 500,000 and preferably from 4000 to 200,000.

As liposoluble film-forming polymers that may be used in the invention, mention may also be made of polyalkylenes and especially copolymers of C₂-C₂₀ alkenes, for instance polybutene, alkylcelluloses with a saturated or unsaturated, linear or branched C₁ to C₈ alkyl radical, for instance ethylcellulose and propylcellulose, vinylpyrrolidone (VP) copolymers and especially copolymers of vinylpyrrolidone and of a C₂ to C₄₀ and better still C₃ to C₂₀ alkene. As examples of VP copolymers that may be used in the invention, mention may be made of the VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene or VP/acrylic acid/lauryl methacrylate copolymer.

The composition according to the invention may also comprise an auxiliariy film-forming agent that promotes the formation of a film with the film-forming polymer. Such a film-forming agent may be chosen from any compound known to the those skilled in the art as being capable of satisfying the desired function, and may be chosen especially from plasticizers and coalescers.

The composition or product of foundation type of the invention is generally in the form of a foundation to be applied especially to the face or the neck, a concealer product, a complexion corrector, a tinted cream or makeup base for the face or a makeup composition for the body.

The composition of foundation type of the invention, especially the base composition and/or the surface composition of the product of foundation type according to the invention, may be in a solid form, for example in pulverulent, compacted or cast form or in the form of a stick or in the form of a fluid, for example a pasty or liquid fluid. It may also be in the form of a soft paste, an ointment or a solid or fluid pomade of cream type. For example, it may be an oil-in-water or water-in-oil emulsion, a gel, especially a solid or soft anhydrous gel, and may even be in two-phase form. According to this variant, it is more particularly in the form of a foundation with an oily and especially anhydrous continuous phase; in this case, it may contain an aqueous phase in a content of less than 5%.

In the case of the product according to the invention, the two corresponding compositions may be in identical or different forms, and especially in accordance with the foregoing text.

The composition of foundation type according to the invention, especially the base composition and/or the surface composition of the product of foundation type according to the invention, may be manufactured by the known processes generally used in cosmetics.

### EXAMPLES

### Examples of formulations of lightening compositions are given below.

The proportions are expressed on a weight basis.

Composition A

| | |
|---|---|
| oxyethylenated polymethylcetyldimethylmethylsiloxane | 0.80% |
| (Abil EM 90 from the company Goldschmidt) | |
| polyglycerol isostearate (4 mol) | 0.60% |
| (Isolan GI 34 from the company Goldschmidt) | |
| hexyl laurate | 0.60% |
| oxyethylenated polydimethylsiloxane | 4.48% |
| (DP: 70 - viscosity: 500 CST) | |
| (KF-6017 from the company Shin Etsu Silidones) | |
| isoeicosane (Permethyl 102 A from Permethyl) | 2% |
| polydimethylsiloxane (viscosity: 5 CST) | 2% |
| (Fluid 200 5 CS from the company Dow Corning) | |
| isostearyl neopentanoate | 0.50% |
| cyclohexadimethylsiloxane | 8% |
| cyclopentadimethylsiloxane | 11.36% |
| isododecane | 13% |
| D,L-α-tocopherol (vitamin E) | 0.08% |
| hectorite modified with distearyldimethylammonium chloride | 1.60% |
| (Bentone 38 V from the company Elementis) | |
| yellow iron oxide coated with perfluoroalkyl phosphate, | 1.86% |
| as a dispersion at 50% by weight in | |
| decamethylcyclopentasiloxane/dimethicone | |
| copolyol (CI: 77492) (FA50DYF from the company Kobo) | |
| brown iron oxide coated with perfluoroalkyl phosphate, | 0.72% |
| as a dispersion at 50% by weight in cyclomethicone/ | |
| dimethylpolysiloxane copolyol (CI: 77491) | |
| (FA50DRF from the company Kobo) | |
| black iron oxide coated with perfluoroalkyl phosphate, | 0.34% |
| as a dispersion at 65% by weight in cyclomethicone/ | |
| dimethylpolysiloxane copolyol (CI: 77499) | |
| (FA65DBF from the company Kobo) | |
| alumina-treated titanium oxide coated with perfluoroalkyl | 7.39% |
| phosphate, as a dispersion at 65% by weight in | |
| decamethylcyclopentasiloxane/ | |
| dimethiconecopolyol (CI 77891) | |
| (FA65DF from the company Kobo) | |
| hollow polymethyl methacrylate microspheres | 4% |
| (particle size: 10 to 12 microns), (Covabead LH 85 | |
| from the company Wackherr) | |
| 1,3-butylene glycol | 10% |
| sodium chloride | 0.70% |
| preserving agents | 0.90% |
| mica-brown iron oxide (60/40) (CI: 77019 + 77491) | 2% |
| (Colorona Passion Orange from the company Merck) | |
| disodium salt of tartrazine (CI: 19140) | 2% |
| (FD & C Yellow 5 from the company LCW) | |
| water | quantity |
| | sufficient |
| | for |
| | (qs) 100% |

Composition B

This is a foundation that is more particularly suitable for light dark skins. Its formulation is as follows:

| | |
|---|---|
| oxyethylenated polymethylcetyldimethylmethylsiloxane | 0.80% |
| (Abil EM 90 from the company Goldschmidt) | |
| polyglycerol isostearate (4 mol) | 0.60% |
| (Isolan GI 34 from the company Goldschmidt) | |
| hexyl laurate | 0.60% |
| oxyethylenated polydimethylsiloxane | 4.48% |
| (DP: 70 - viscosity: 500 CST) | |
| (KF-6017 from the company Shin Etsu Silicones) | |
| isoeicosane (Permethyl 102 A from Permethyl) | 2% |
| polydimethylsiloxane (viscosity: 5 CST) | 2% |
| (Fluid 200 5 CS from the company Dow Coming) | |
| isostearyl neopentanoate | 0.50% |
| cyclohexadimethylsiloxane | 8% |
| cyclopentadimethylsiloxane | 11.36% |
| isododecane | 13% |
| D,L-α-tocopherol (vitamin E) | 0.08% |
| hectorite modified with distearyldimethylammonium chloride | 1.60% |
| (Bentone 38 V from the company Elementis) | |
| yellow iron oxide coated with perfluoroalkyl phosphate, | 6.90% |
| as a dispersion at 50% by weight in | |
| decamethylcyclopentasiloxane/dimethicone | |
| copolyol (CI: 77492) (FA50DYF from the company Kobo) | |
| mica-bismuth oxychloride-brown iron oxide (47/28/25) | 6.81% |
| (Chroma —Lite Brown from the company Engelhard) | |
| aluminium lake of red Allura on alumina (40/60) | 0.60% |
| (FD&C Red 40 Al lake from the company Noveon) | |
| hollow polymethyl methacrylate microspheres | 4% |
| (particle size: 10 to 12 microns), (Covabead LH 85 from | |
| the company Wackherr) | |
| 1,3-butylene glycol | 10% |
| sodium chloride | 0.70% |
| preserving agents | 0.90% |
| water | qs 100% |

Composition C

This composition is most particularly suitable for black skins. Its formulation is as follows:

| | |
|---|---|
| oxyethylenated polymethylcetyldimethylmethylsiloxane | 0.80% |
| (Abil EM 90 from the company Goldschmidt) | |
| polyglycerol isostearate (4 mol) | 0.60% |
| (Isolan GI 34 from the company Goldschmidt) | |
| hexyl laurate | 0.60% |
| oxyethylenated polydimethylsiloxane | 4.48% |
| (DP: 70 - viscosity: 500 CST) | |
| (KF-6017 from the company Shin Etsu Silicones) | |
| isoeicosane (Permethyl 102 A) | 2% |
| polydimethylsiloxane (viscosity: 5 CST) | 2% |
| (Fluid 200 5 CS from the company Dow Corning) | |
| isostearyl neopentanoate | 0.50% |
| cyclohexadimethylsiloxane | 8% |
| cyclopentadimethylsiloxane | 11.36% |
| isododecane | 13% |
| D,L-α-tocopherol (vitamin E) | 0.08% |
| hectorite modified with distearyldimethylammonium chloride | 1.60% |
| (Bentone 38V from the company Elementis) | |
| yellow iron oxide coated with perfluoroalkyl phosphate, | 1.86% |
| as a dispersion at 50% by weight in | |
| decamethylcyclopentasiloxane/dimethicone | |
| copolyol (CI: 77492) (FA50DYF from the company Kobo) | |
| brown iron oxide coated with perfluoroalkyl phosphate, | 0.72% |
| as a dispersion at 50% by weight in | |
| cyclomethicone/dimethylpolysiloxane | |
| copolyol (CI: 77491) (FA50DRF from the company Kobo) | |
| black iron oxide coated with perfluoroalkyl phosphate, | 0.34% |
| as a dispersion at 65% by weight in | |
| cyclomethicone/dimethylpolysiloxane | |
| copolyol (CI: 77499) (FA65DBF from the company Kobo) | |
| alumina-treated titanium oxide coated with perfluoroalkyl | 7.39% |
| phosphate, as a dispersion at 65% by weight in | |
| decamethylcyclopentasiloxane/ | |
| dimethiconecopolyol (CI 77891) | |
| (FA65DF from the company Kobo) | |
| hollow polymethyl methacrylate microspheres | 4% |
| (particle size: 10 to 12 microns), (Covabead LH 85 | |
| from the company Wackherr) | |
| 1,3-butylene glycol | 10% |
| sodium chloride | 0.70% |
| preserving agents | 0.90% |
| silica-brown iron oxide (Xirona Indian Summer from Merck) | 4% |
| water | qs 100% |

Composition D

This is a foundation composition that is more particularly suitable for black skins. Its formulation is as follows:

| | |
|---|---|
| oxyethylenated polymethylcetyldimethylmethylsiloxane | 0.80% |
| (Abil EM 90 from the company Goldschmidt) | |
| polyglycerol isostearate (4 mol) | 0.60% |
| (Isolan GI 34 from the company Goldschmidt) | |
| hexyl laurate | 0.60% |
| oxyethylenated polydimethylsiloxane | 4.48% |
| (DP: 70 - viscosity: 500 CST) | |
| (KF-6017 from the company Shin Etsu Silicones) | |
| isoeicosane (Permethyl 102 A) | 2% |
| polydimethylsiloxane (viscosity: 5 CST) | 2% |
| (Fluid 200 5 CS from the company Dow Corning) | |
| isostearyl neopentanoate | 0.50% |
| cyclohexadimethylsiloxane | 8% |
| cyclopentadimethylsiloxane | 11.36% |
| isododecane | 13% |
| D,L-α-tocopherol (vitamin E) | 0.08% |
| hectorite modified with distearyldimethylammonium chloride | 1.60% |
| (Bentone 38 V from the company Elementis) | |
| yellow iron oxide coated with perfluoroalkyl phosphate, | 5.25% |
| as a dispersion at 50% by weight in | |
| decamethylcyclopentasiloxane/dimethicone | |
| copolyol (CI: 77492) (FA50DYF from the company Kobo) | |
| brown iron oxide coated with perfluoroalkyl phosphate, | 0.72% |
| as a dispersion at 50% by weight in | |
| cyclomethicone/dimethylpolysiloxane | |
| copolyol (CI: 77491) (FA50DRF from the company Kobo) | |
| black iron oxide coated with perfluoroalkyl phosphate, | 0.34% |
| as a dispersion at 65% by weight in | |
| cyclomethicone/dimethylpolysiloxane | |
| copolyol (CI: 77491) (FA65DBF from the company Kobo) | |
| alumina-treated titanium oxide coated with perfluoroalkyl | 4% |
| phosphate, as a dispersion at 65% by weight in | |
| decamethylcyclopentasiloxane/dimethiconecopolyol | |
| (CI 77891) (FA65DF from the company Kobo) | |
| hollow polymethyl methacrylate microspheres | 4% |
| (particle size: 10 to 12 microns), (Covabead LH 85 | |
| from the company Wackherr) | |
| 1,3-butylene glycol | 10% |
| sodium chloride | 0.70% |
| silica-brown iron oxide (Xirona Indian Summer from Merck) | 4% |
| preserving agents | 0.90% |
| water | qs 100% |

Composition E

This is a stick for black skin. Its formulation is as follows:

| | |
|---|---|
| polyethylene wax (MW: 500) (Polywax 500 from Bareco) | 4% |
| ethylene homopolymer (melting point: 79.5° C.) | 8% |
| (Performalene 400 from New Phase Technologies) | |
| cyclopentadimethylsiloxane | 5% |
| cyclohexadimethylsiloxane | 20% |
| isododecane | 19% |
| phenyl trimethylsiloxy trisiloxane (viscosity: 20 CST - PM: 372) | 19% |
| (DC556 from Dow Corning) | |
| hollow polymethyl methacrylate microspheres | 10% |
| (particle size: 10 to 12 microns) (Covabead LH85 from Wackherr) | |
| mica-brown iron oxide (94/6) (CI: 77019 + 77491) | 12% |
| (Cosmetica Orange from Engelhard) | |
| yellow iron oxide coated with perfluoroalkyl phosphate, | 3% |
| as a dispersion at 50% by weight in decamethylcyclopentasiloxane/ | |
| dimethicone copolyol (CI: 77492) | |
| (FA50DYF from the company Kobo) | |

Composition F

This is a fluid foundation for black skins. Its formulation is as follows:

| | |
|---|---|
| cetyl polyethylene glycol/PPG - 10/1-dimethicone | 0.80% |
| (Abil EM 90) | |
| polyglyceryl 4-isostearate (Isolan G134 from the company | 0.60% |
| Goldschmidt) | |
| hexyl laurate | 0.60% |
| PEG-10 dimethicone (KF6017) | 4.48% |
| isoeicosane (Permethyl 102A) | 2% |
| dimethicone (DC 200 Fluid) | 2% |
| isostearyl neopentanoate | 0.50% |
| cyclohexasiloxane | 8% |
| cyclopentasiloxane | 11.36% |
| isododecane | 13% |
| D, L-α-tocopherol (vitamin E) | 0.08% |
| hectorite modified with distearyldimethylammonium chloride | 1.6% |
| (Bentone 38V from the company Elementis) | |
| iron oxides and cyclopentasiloxane and PEG/PPG-18/ | 4.73% |
| 18-dimethicone and C₉ to C₁₅ fluoroalkyl | |
| phosphates (FA5ODYF from Kobo) | |
| iron oxides and cyclopentasiloxane and PEG/PPG-18/ | 2.595% |
| 18-dimethicone and C₉ to C₁₅ fluoroalkyl | |
| phosphates (FA50DRF from Kobo) | |
| iron oxides and cyclopentasiloxane and PEG/PPG-18/ | 1.22% |
| 18-dimethicone and C₉ to C₁₅ fluoroalkyl | |
| phosphates (FA65DBF from Kobo) | |
| titanium dioxide and cyclopentasiloxane and PEG/PPG-18/ | 1.765% |
| 18-dimethicone and C₉ to C₁₅ fluoroalkyl | |
| phosphates and alumina (FA65DF from Kobo) | |
| polymethyl methacrylate (Covabead LH 85) | 4% |
| butylene glycol | 10% |
| sodium chloride | 0.70% |
| preserving agents | 0.90% |
| mica and iron oxides (Colorona Passion Orange from Merck) | 2% |
| Yellow-6 lake (pigments: FDC Yellow 6 A1 Lake from Sun | 2% |
| Chemical) (CI15985) | |
| water | qs 100% |

Composition Y

This is a foundation that is more particularly intended for dark skins. Its formulation is as follows:

| | |
|---|---|
| oxyethylenated polymethylcetyldimethylmethylsiloxane | 0.80% |
| (Abil EM 90 from the company Goldschmidt) | |
| polyglycerol isostearate (4 mol) | 0.60% |
| (Isolan GI 34 from the company Goldschmidt) | |
| hexyl laurate | 0.60% |
| oxyethylenated polydimethylsiloxane (DP: 70 - viscosity: | 4.48% |
| 500 CST) (KF-6017 from the company Shin Etsu Silidones) | |
| isoeicosane (Permethyl 102 A from Permethyl) | 2% |
| polydimethylsiloxane (viscosity: 5 CST) | 2% |
| (Fluid 200 5 CS from the company Dow Corning) | |
| isostearyl neopentanoate | 0.50% |
| cyclohexadimethylsiloxane | 8% |
| cyclopentadimethylsiloxane | 11.3% |
| isododecane | 13% |
| D,L-α-tocopherol (vitamin E) | 0.08% |
| hectorite modified with distearyldimethylammonium chloride | 1.60% |
| (Bentone 38V from the company Elementis) | |
| yellow iron oxide coated with perfluoroalkyl phosphate, | 10.93% |
| as a dispersion at 50% by weight in | |
| decamethylcyclopentasiloxane/dimethicone copolyol | |
| (CI: 77492) (FA50DYF from the company Kobo) | |
| brown iron oxide coated with perfluoroalkyl phosphate, | 2.15% |
| as a dispersion at 50% by weight in cyclomethicone/ | |
| dimethylpolysiloxane copolyol (CI: 77491) (FA50DRF | |
| from the company Kobo) | |
| hollow polymethyl methacrylate microspheres | 4% |
| (particle size: 10 to 12 microns) (Covabead LH 85 | |
| from Wackherr) | |
| mica-ferric blue sold under the name | 1.23% |
| Microna Matte Blue by the company Merck | |
| 1,3-butylene glycol | 10% |
| sodium chloride | 0.70% |
| preserving agents | 0.90% |
| water | qs 100% |

Composition Z

This is a foundation for dark skins. Its composition is as follows:

| | |
|---|---|
| oxyethylenated polymethylcetyldimethylmethylsiloxane | 0.80% |
| (Abil EM 90 from the company Goldschmidt) | |
| polyglycerol isostearate (4 mol) | 0.60% |
| (Isolan GI 34 from the company Goldschmidt) | |
| hexyl laurate | 0.60% |
| oxyethylenated polydimethylsiloxane (DP: 70 - viscosity: | 4.70% |
| 500 CST) (KF-6017 from the company Shin Etsu Silicones) | |
| isoeicosane (Permethyl 102 A from Permethyl) | 2% |
| polydimethylsiloxane (viscosity: 5 CST) | 2% |
| (Fluid 200 5 CST from the company Dow Corning) | |
| isostearyl neopentanoate | 0.50% |
| cyclohexadimethylsiloxane | 8.4% |
| cyclopentadimethylsiloxane | 11.90% |
| isododecane | 13.60% |
| D,L-α-tocopherol (vitamin E) | 0.08% |
| hectorite modified with distearyldimethylammonium chloride | 1.67% |
| (Bentone 38V from the company Elementis) | |
| yellow iron oxide coated with perfluoroalkyl phosphate, | 4.73% |
| as a dispersion at 50% by weight in | |
| decamethylcyclopentasiloxane/dimethicone copolyol | |
| (CI: 77492) (FA50DYF from the company Kobo) | |
| brown iron oxide coated with perfluoroalkyl phosphate, | 2.60% |
| as a dispersion at 50% by weight in cyclomethicone/ | |
| dimethylpolysiloxane copolyol (CI: 77491) (FA50DRF | |
| from the company Kobo) | |
| black iron oxide coated with perfluoroalkyl phosphate, | 1.20% |
| as a dispersion at 65% by weight in cyclomethicone/ | |
| dimethylpolysiloxane copolyol (CI: 77499) (FA65DBF | |
| from the company Kobo) | |
| alumina-treated titanium oxide coated with perfluoroalkyl | 1.20% |
| phosphate, as a dispersion at 65% by weight in | |
| decamethylcyclopentasiloxane/dimethiconecopolyol | |
| (CI 77891) (FA65DF from the company Kobo) | |
| hollow polymethyl methacrylate microspheres | 4% |
| (particle size: 10 to 12 microns), (Covabead LH 85 | |
| from the company Wackherr) | |
| 1,3-butylene glycol | 10% |
| sodium chloride | 0.70% |
| preserving agents | 0.90% |
| water | qs 100% |

Composition G

| | |
|---|---|
| oxyethylenated polymethylcetyldimethylmethylsiloxane | 0.80% |
| (Abil EM 90 from the company Goldschmidt) | |
| polyglycerol isostearate (4 mol) | 0.60% |
| (Isolan GI 34 from the company Goldschmidt) | |
| hexyl laurate | 0.60% |
| oxyethylenated polydimethylsiloxane (DP 70 - viscosity: | 4.48% |
| 500 CST) (KF-6017 from the company Shin Etsu Silidones) | |
| isoeicosane (Permethyl 102 A from Permethyl) | 2% |
| polydimethylsiloxane (viscosity: 5 CST) | 2% |
| (FLuid 200 5 CS from the company Dow Corning) | |
| isostearyl neopentanoate | 0.5% |
| cyclohexadimethylsiloxane | 8% |
| isododecane | 13% |
| D,L-α-tocopherol (vitamin E) | 0.08% |
| Smectite: modified magnesium silicate in | 12.96% |
| cyclopentadimethylsiloxane and ethanol (Bentone VS 5 V gel) | |
| yellow iron oxide coated with perfluoroalkyl phosphate | 1.28% |
| (PF 5 Yellow 601 from Daito) | |
| brown iron oxide coated with perfluoroalkyl phosphate | 2.13% |
| (PF 5 Red R 516 L from Daito) | |
| black iron oxide coated with perfluoroalkyl phosphate | 0.47% |
| (PF 5 Black BL 100 from Daito) | |
| ultramarine blue coated with perfluoroalkyl phosphate | 1.28% |
| (PF 5 Ultramarine No. 801 from Daito) | |
| cyclopentadimethylsiloxane (Dow Corning 245 Fluid | 5.17% |
| from Dow Corning) | |
| hollow polymethyl methacrylate microspheres | 4% |
| (particle size: 10 to 12 μm), (Covabead LH85 | |
| from the company Wackherr) | |
| 1,3-butylene glycol | 10% |
| sodium chloride | 0.7% |
| preserving agents | 0.90% |
| phenoxyethanol (Phenoxetol) | 0.5% |
| mica-brown iron oxide (60/40) (CI: 77019 + 77491) | 2% |
| (Colorona Passion Orange from the company Merck) | |
| Yellow-6 lake (pigments: FDC Yellow 6 A1 Lake from Sun | 2% |
| Chemicals) (CI15985) | |
| water | qs 100% |

Composition H

| | |
|---|---|
| oxyethylenated polymethylcetyldimethylmethylsiloxane | 0.80% |
| (Abil EM 90 from the company Goldschmidt) | |
| polyglycerol isostearate (4 mol) | 0.60% |
| (Isolan GI 34 from the company Goldschmidt) | |
| hexyl laurate | 0.60% |
| oxyethylenated polydimethylsiloxane (DP: 70 - viscosity: | 4.48% |
| 500 CST) (KF-6017 from the company Shin Etsu Silicones) | |
| isoeicosane (Permethyl 102 A from Permethyl) | 2% |
| polydimethylsiloxane (viscosity: 5 CST) | 2% |
| (FLuid 200 5 CS from the company Dow Corning) | |
| isostearyl neopentanoate | 0.5% |
| cyclohexadimethylsiloxane | 8% |
| isododecane | 13% |
| D,L-α-tocopherol (vitamin E) | 0.08% |
| Smectite: modified magnesium silicate in | 12.96% |
| cyclopentadimethylsiloxane and ethanol (Bentone VS 5 V gel) | |
| yellow iron oxide coated with perfluoroalkyl phosphate | 2.37% |
| (PF 5 Yellow 601 from Daito) | |
| brown iron oxide coated with perfluoroalkyl phosphate | 1.3% |
| (PF 5 Red R 516 L from Daito) | |
| black iron oxide coated with perfluoroalkyl phosphate | 0.61% |
| (PF 5 Black BL 100 from Daito) | |
| titanium oxide coated with perfluoroalkyl phosphate | 0.88% |
| (PF 5 Ultramarine No. 801 from Daito) | |
| cyclopentadimethylsiloxane (Dow Corning 245 Fluid | 5.13% |
| from Dow Corning) | |
| hollow polymethyl methacrylate microspheres | 4% |
| (particle size: 10 to 12 μm), (Covabead LH85 from | |
| the company Wackherr) | |
| 1,3-butylene glycol | 10% |
| sodium chloride | 0.7% |
| preserving agents | 0.90% |
| phenoxyethanol (Phenoxetol) | 0.5% |
| mica-brown iron oxide (60/40) (CI: 77019 + 77491) | 2% |
| (Colorona Passion Orange from the company Merck) | |
| Yellow-6 lake (pigments: FDC Yellow 6 A1 Lake from Sun | 2% |
| Chemicals) (CI15985) | |
| water | qs 100% |

Composition I

| | |
|---|---|
| oxyethylenated polymethylcetyldimethylmethylsiloxane | 0.80% |
| (Abil EM 90 from the company Goldschmidt) | |
| polyglycerol isostearate (4 mol) | 0.60% |
| (Isolan GI 34 from the company Goldschmidt) | |
| hexyl laurate | 0.60% |
| oxyethylenated polydimethylsiloxane (DP: 70 - viscosity: | 4.48% |
| 500 CST) (KF-6017 from the company Shin Etsu Silicones) | |
| isoeicosane (Permethyl 102 A from Permethyl) | 2% |
| polydimethylsiloxane (viscosity: 5 CST) | 2% |
| (Fluid 200 5 CS from the company Dow Corning) | |
| isostearyl neopentanoate | 0.5% |
| cyclohexadimethylsiloxane | 8% |
| isododecane | 13% |
| D,L-α-tocopherol (vitamin E) | 0.08% |
| Smectite: modified magnesium silicate in | 12.96% |
| cyclopentadimethylsiloxane and ethanol (Bentone VS 5 V gel) | |
| yellow iron oxide coated with perfluoroalkyl phosphate | 3.46% |
| (PF 5 Yellow 601 from Daito) | |
| cyclopentadimethylsiloxane (Dow Corning 245 Fluid | 3.46% |
| from Dow Corning) | |
| hollow polymethyl methacrylate microspheres | 4% |
| (particle size: 10 to 12 μm), (Covabead LH85 from | |
| the company Wackherr) | |
| 1,3-butylene glycol | 10% |
| sodium chloride | 0.7% |
| preserving agents | 0.90% |
| phenoxyethanol (Phenoxetol) | 0.5% |
| mica-bismuth oxychloride brown iron oxide | 6.81% |
| (Chroma-Lite Brown 4509 from Engelhard) | |
| aluminium lake of red allura on alumina | 0.6% |
| (FDC Red 40 AL Lake - 6808 from Noveon) | |
| water | qs 100% |

Composition J

| | |
|---|---|
| oxyethylenated polymethylcetyldimethylmethylsiloxane | 0.80% |
| (Abil EM 90 from the company Goldschmidt) | |
| polyglycerol isostearate (4 mol) | 0.60% |
| (Isolan GI 34 from the company Goldschmidt) | |
| hexyl laurate | 0.60% |
| oxyethylenated polydimethylsiloxane (DP: 70 - viscosity: | 4.48% |
| 500 CST) (KF-6017 from the company Shin Etsu Silicones) | |
| isoeicosane (Permethyl 102 A from Permethyl) | 2% |
| polydimethylsiloxane (viscosity: 5 CST) | 2% |
| (FLuid 200 5 CS from the company Dow Corning) | |
| isostearyl neopentanoate | 0.5% |
| cyclohexadimethylsiloxane | 8% |
| isododecane | 13% |
| D,L-α-tocopherol (vitamin E) | 0.08% |
| Smectite: modified magnesium silicate in | 12.96% |
| cyclopentadimethylsiloxane and ethanol (Bentone VS 5 V gel) | |
| yellow iron oxide coated with perfluoroalkyl phosphate | 1.75% |
| (PF 5 Yellow 601 from Daito) | |
| brown iron oxide coated with perfluoroalkyl phosphate | 0.39% |
| (PF 5 Red R 516 L from Daito) | |
| black iron oxide coated with perfluoroalkyl phosphate | 0.14% |
| (PF 5 Black BL 100 from Daito) | |
| titanium oxide coated with perfluoroalkyl phosphate | 2.22% |
| (PF 5 TiO2 A 100 from Daito) | |
| cyclopentadimethylsiloxane (Dow Corning 245 Fluid | 4.49% |
| from Dow Coming) | |
| hollow polymethyl methacrylate microspheres | 4% |
| (particle size: 10 to 12 μm), (Covabead LH85 from | |
| the company Wackherr) | |
| 1,3-butylene glycol | 10% |
| sodium chloride | 0.7% |
| preserving agents | 0.90% |
| phenoxyethanol (Phenoxetol) | 0.5% |
| silica-brown iron oxide (Xirona Indian Summer | 3% |
| from Merck) | |
| mica-bismuth oxychloride-yellow iron oxide | 2.32% |
| (Chroma-Lite Yellow 4502 from Engelhard) | |
| water | qs 100% |

Composition K

| | |
|---|---|
| oxyethylenated polymethylcetyldimethylmethylsiloxane | 0.80% |
| (Abil EM 90 from the company Goldschmidt) | |
| polyglycerol isostearate (4 mol) | 0.60% |
| (Isolan GI 34 from the company Goldschmidt) | |
| hexyl laurate | 0.60% |
| oxyethylenated polydimethylsiloxane (DP: 70 - viscosity: | 4.48% |
| 500 CST) (KF-6017 from the company Shin Etsu Silicones) | |
| isoeicosane (Permethyl 102 A from Permethyl) | 2% |
| polydimethylsiloxane (viscosity: 5 CST) | 2% |
| (Fluid 200 5 CS from the company Dow Corning) | |
| isostearyl neopentanoate | 0.5% |
| cyclohexadimethylsiloxane | 8% |
| isododecane | 13% |
| D,L-α-tocopherol (vitamin E) | 0.08% |
| Smectite: modified magnesium silicate in | 12.96% |
| cyclopentadimethylsiloxane and ethanol (Bentone VS 5 V gel) | |
| yellow iron oxide coated with perfluoroalkyl phosphate | 1.75% |
| (PF 5 Yellow 601 from Daito) | |
| brown iron oxide coated with perfluoroalkyl phosphate | 0.39% |
| (PF 5 Red R 516 L from Daito) | |
| black iron oxide coated with perfluoroalkyl phosphate | 0.19% |
| (PF 5 Black BL 100 from Daito) | |
| titanium oxide coated with perfluoroalkyl phosphate | 2.17% |
| (PF 5 TiO2 A 100 from Daito) | |
| cyclopentadimethylsiloxane (Dow Corning 245 Fluid | 4.49% |
| from Dow Corning) | |
| hollow polymethyl methacrylate microspheres | 4% |
| (particle size: 10 to 12 μm), (Covabead LH85 from | |
| the company Wackherr) | |
| 1,3-butylene glycol | 10% |
| sodium chloride | 0.7% |
| preserving agents | 0.90% |
| phenoxyethanol (Phenoxetol) | 0.5% |
| silica-brown iron oxide (Xirona Indian Summer | 3.5% |
| from Merck) | |
| mica-bismuth oxychloride-yellow iron oxide | 1.83% |
| (Chroma-Lite Yellow 4502 from Engelhard) | |
| water | qs 100% |

Measurement of the Color of the Compositions

The color of each composition A, B, C, Y, Z, G, H, I, J, K is given in the table below, the values being indicated with a precision of 15%, better still 10% and even better still 5%:

| Composition | L* | C* | h | Interval |
|---|---|---|---|---|
| A | 39.46 | 41.52 | 63.34° | 30 < L* < 40 |
| B | 48.59 | 37.77 | 57.65° | 40 < L* < 50 |
| C | 57.24 | 24.53 | 53.22° | 50 < L* < 60 |
| Y | 39.22 | 35.08 | 66.83° | 30 < L* < 40 |
| Z | 38.21 | 31.39 | 55.34° | 30 < L* < 40 |
| G | 38.04 | 23.06 | 42.08° | 30 < L* < 40 |
| H | 43.50 | 29.31 | 46.30° | 40 < L* < 50 |
| I | 48.38 | 30.06 | 51.46° | 40 < L* < 50 |
| J | 55.89 | 29.17 | 53° | 50 < L* < 60 |
| K | 55.78 | 27.32 | 51.93° | 50 < L* < 60 |

To measure the color of a composition, a color measurement in the bulk of the product was performed, as follows.

A cuvette referenced H247 was filled with the composition.

The cuvette was levelled off and a glass slide was then applied onto the composition, taking care to avoid air bubbles under the slide. A color measurement was taken using a Minolta® spectrocolorimeter of CM-2002 type with medium aperture and specular reflection mode included.

The compositions A, Y, Z and G have a lightness of between 30 and 40, and are more particularly suitable for dark skins of dark-tone complexion.

Compositions B, H and I have a lightness of between 40 and 50 and are more particularly suitable for dark skins of medium-tone complexion.

Finally, compositions C, J and K have a lightness of between 50 and 60 and are suitable for dark skins of fair-tone complexion.

A spectral reflectance measurement was also performed, in the bulk, using a Minolta® spectrocolorimeter of type CM3700d, in reflection mode, specular excluded, UV included, and with a small aperture d/8.

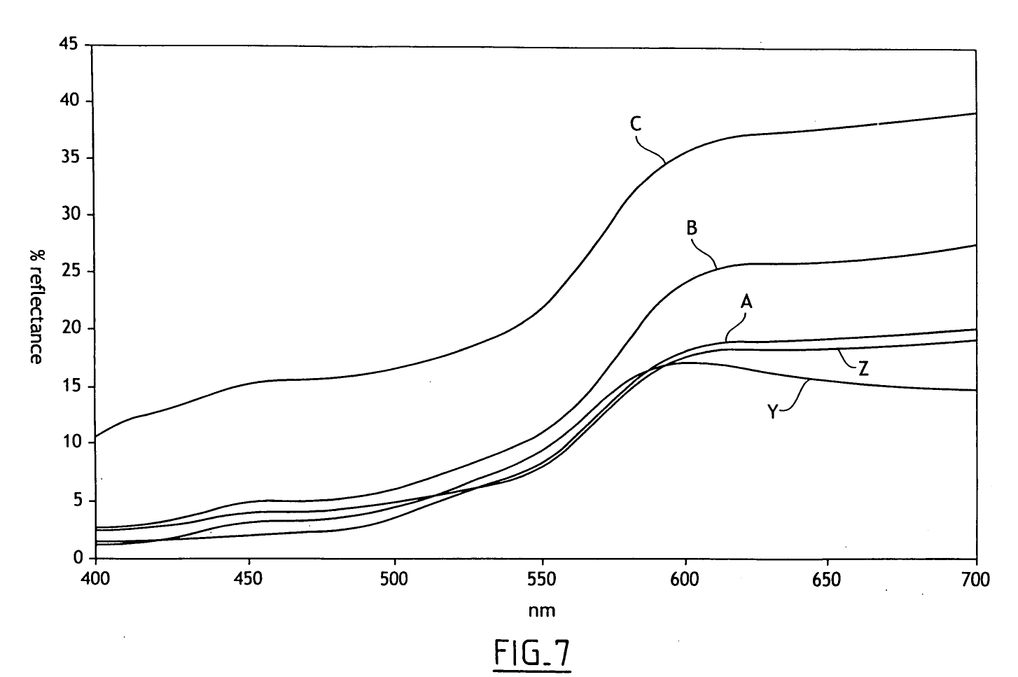

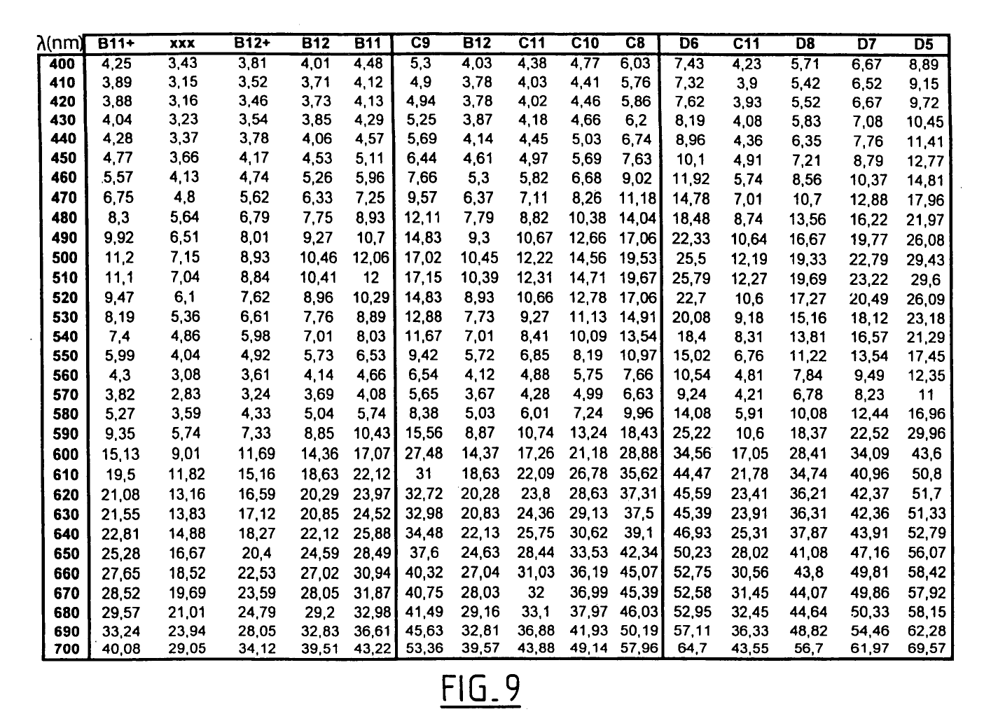

FIG. 7 shows the reflectance spectra of compositions A, B, C, Y and Z present in metal dishes with a side length of a few centimetres, the thickness of the composition being of the order of one centimetre.

It may be noted that, for the wavelength region of between 600 and 680 nm, the reflectance is between 10 and 45% and more specifically between 12 and 40% for the five test compositions. The reflectance is less than 20% in the range from 450 to 500 nm.

In Vivo Tests

Measurements may be taken before and after applying makeup, for example using the acquisition device described with reference to FIG. 1, as described above.

Dark skins having a complexion defined by a lightness L* of between 35 and 42, a saturation C* of between 13 and 20, a position a* on the red/green axis of between 9 and 15, a position b* on the blue/yellow axis of between 13 and 17 and a hue angle value h of between 39° and 52°, were made up with composition A.

For an individual with a lightness of 35.5 and a saturation of 15.3, lightness and saturation values after applying makeup of 37.6 and 16.5, respectively, i.e a lightness variation ΔL* of 2.1 and a saturation variation ΔC* of 1.2, were able to be measured. Other tests showed that composition A appeared to be entirely suitable for lightening skins whose lightness L* was less than 42 and saturation C* less than 21.

Composition B was applied to an individual having a lightness of 44.4 and a saturation of 19.8. Differences ΔL* and ΔC* of 0.8 and 0.6, respectively, were measured and a satisfactory result was obtained.

Composition C is more suitable for fair-tone dark skins, especially those with a lightness of between 48 and 54 and a saturation of between 24 and 28.

It was also possible to obtain a lightening makeup result with compositions Y and Z.

Tests on Contrast Cards

In order to determine the homogenization power 1/ΔE₁ and the covering power 1/ΔE₂ of a composition, the contrast cards described above may be used.

Using the contrast cards 30, 31 and 32, the various compositions A, B, C, Y, Z, G, I, J were able to be characterized by proceeding in the following manner.

Liquid or Pasty Compositions

A coat of foundation 30 μm thick is applied with an automatic spreader of Braive Instruments type, over a Folex film (Copier Film) (of reference 117230).

The foundation-coated film is dried in an oven at 37° C. for 24 hours and is then superposed on one of the contrast cards 30, 31 or 32 depending on the lightness of the foundation.

Preferably, the contrast card 32 is covered with a composition suitable for dark-tone dark complexions, the contrast card 31 is covered with a composition suitable for medium-tone complexions, and the card 30 is covered with a composition suitable for fair-tone complexions.

Thus, compositions A, Y, Z and G were able to be characterized using the contrast card 32.

Compositions B, H and I were moreover able to be characterized using the contrast card 31.

Finally, compositions C, J and K were able to be characterized using the contrast card 30.

For the compositions suitable for dark-tone dark complexions, the colorimetric difference ΔE_{1 zone/B11+} between, on the one hand, each zone B11, B12, B12+, XXX and, on the other hand, zone B11+, is measured through the film coated with composition and superposed on the contrast card 32, and the mean difference ΔE₁ mean is then calculated:
ΔE_{1 mean}=(ΔE_{1 B11/B11}++ΔE_{1 B12/B11+}+ΔE_{1 B12+/B11+}+ΔE_{1 XXX/B11+})/4
with
ΔE_{1B11/B11+}=[(a*_{B11}−a*_{B11+})²+(b*_{B11-b*B}₁₁₊)²+(L*_{B11}−L*_{B11+})²]^{1/2}

In general the following notation will be used:
ΔE_{1 M/N}=[(a_{M}*−a_{N}*)²+(b_{M}*−b_{N}*)²+(L_{M}*−L_{N}*)²]^{1/2}

The homogenization power is defined by 1/ΔE_{1 mean}.

The colorimetric difference ΔE₂ between zone B11+ and the white border 34 is also measured and the covering power is defined by 1/ΔE₂.

The following result was obtained with compositions A, Y, Z and G:

| Composition | A | Y | Z | G |
|---|---|---|---|---|
| ΔE_{1 mean} | 0.24 | 0.36 | 0.62 | 0.68 |
| ΔE₂ | 1.58 | 1.67 | 1.84 | 3.98 |

It may especially be sought, using the contrast card, to select the compositions so as to have high homogenization power without the covering power being too high. High homogenization power can make it possible to mask skin defects. A covering power that is not too high can allow the skin to conserve a natural appearance.

For the compositions suitable for medium-tone complexions, the film is superposed on the contrast card 31.

The colorimetric difference ΔE_{1 zone/C9} between, on the one hand, each zone C8, C10, C11 and B12 and, on the other hand, zone C9, is measured through the film thus coated with composition, and the mean difference ΔE_{1 mean} is then calculated:
ΔE_{1 mean}=(ΔE_{1 C8/C9}+ΔE_{1 C10/C9}+ΔE_{1 C11/C9}+ΔE_{B12/C9})/⁴.

The colorimetric difference ΔE₂ between zone C9 and the white border 34 is also measured, and the covering power 1/ΔE₂ is defined.

The following results were obtained with compositions B and I:

Composition

| | B | I |
|---|---|---|
| ΔE₁ₘₑₐₙ | 0.89 | 0.73 |
| ΔE₂ | 8.29 | 6.68 |

For compositions that are suitable for fair-tone dark complexions, the contrast card 30 is used and the colorimetric difference ΔE_{1 zone/D6} between, on the one hand, each zone D5, D7, D8 and C11 and, on the other hand, zone D6, is measured, and the mean difference ΔE₁ mean is then calculated:
ΔE_{1 mean}=(ΔE_{1 D5/D6+}ΔE_{1 D7/D6+}ΔE_{1 D8/D6+}ΔE_{1 C1/D6})/4.

The colorimetric difference ΔE₂ between zone D6 and the white border 34 is also measured, and the covering power 1/ΔE₂ is defined.

The following results were obtained with compositions C and J:

Composition

| | C | J |
|---|---|---|
| ΔE₁ₘₑₐₙ | 0.77 | 1.07 |
| ΔE₂ | 2.96 | 6.68 |

Moreover, using the card shown in FIG. 5, the various compositions A, B, C, Y and Z were able to be characterized in the following manner.

The foundation is applied onto a transparent film with a thickness of 20 μm, it is left to dry for 10 minutes at 37° C. in an oven, and this film is superposed onto the contrast card. The colorimetric difference ΔE_{1 zone z1/z6} between, on the one hand, each zone Z1, Z2, Z3, Z4 and Z5 and, on the other hand, zone Z6, is measured through the film thus coated with composition, and the mean difference ΔE_{1 mean} is then calculated:
ΔE_{1 mean}=(ΔE_{1 zone Z1/Z6+}ΔE_{1 zone Z2/Z6+}ΔE_{1 zone Z3/Z6+}ΔE_{1 zone Z4/Z6+}ΔE_{1 zone z5/z6})/5
with
ΔE_{1 zone Z1/Z6}=[(a*_{Zi}−a*_{Z6})²+(b*_{Zi}−b*_{Z6})²+(L*_{Zi}−L*_{Z6})²]^{1/2}, for i=1, 2, . . . , 5.
The homogenization power is defined by 1/ΔE_{1 mean.}

The colorimetric difference ΔE₂ between the reference zone Z6 and the white border Z7 is also measured and the covering power is defined by 1/ΔE₂.

The following results were obtained with compositions A, B, C, Y and Z:

| | Composition | | | | |
|---|---|---|---|---|---|
| | A | B | C | Y | Z |
| ΔE₁ₘₑₐₙ | 1.85 | 2.80 | 1.97 | 2.00 | 1.88 |
| ΔE₂ | 13.56 | 18.73 | 11.11 | 14.98 | 13.92 |

Other Presentation Forms

Powders

In the case of compacted powders, they are decompacted so as to convert them into free powders.

The powder is applied uniformly onto a flat surface and an adhesive transparent plastic film is then applied over the powder with a pressure of 100 g/cm², such that the powder sticks to the adhesive and to obtain an adhesive surface saturated with powder. The adhesive face charged with powder is then placed against a transparent glass plate and the assembly is applied onto the contrast card. The color measurements for the various zones of the contrast card are taken as previously, through the transparent film charged with composition.

Stick

The stick may be melted so as to be applied in the form of a coat 20 μm thick onto a transparent film. It is allowed to dry for 10 minutes at 37° C. in an oven and the film is then applied to the contrast card, in the manner described above.

Spray

A thickness of 20 μm of composition is applied and is left to dry for 10 minutes at 37° C. in an oven.

Colored Indicator on Packaging

It is advantageously possible to present on packaging, as illustrated in FIG. 8, containing the composition, for example a cardboard case containing a container filled with the composition or the container itself, a colored indicator 20 intended to allow the consumer to determine at the point of sale whether the composition is suitable for his complexion.

The colored indicator 20 may comprise, for example, one or more colored zones 21, 22 and 23 imitating one or more mean colors of skins for which the composition is particularly suitable, for example fair-tone dark skin, medium-tone dark skin or dark-tone dark skin.

At least one of the zones 21, 22 and 23 may have, for example, a lightness L* of between 30 and 60, especially between 30 and 55, a saturation C* of between 8 and 30, especially between 10 and 30 and especially between 12 and 28, and a hue angle value h of between 38° and 63° and especially between 38° and 54°.

A system comprising a sensor may also be provided at the point of sale, this system being arranged, firstly, to measure at least the lightness of the skin, and preferably also its saturation, and secondly to give advice relating to the choice of composition allowing the skin to be lightened. The system may especially be arranged to recommend at least one composition for obtaining a difference in lightness AL and a difference in saturation ΔC*, taking into account the lightness L* and saturation C* of the skin to be made up, such that the following are obtained:
0.5≦ΔL*≦4, preferably 0.5≦ΔL*≦2.5 and
0.5≦ΔC*≦4, preferably 0.5≦ΔC≦2.5.

Throughout the description, including the claims, the expression “comprising one” should be understood as being synonymous with “comprising at least one”, unless otherwise specified.

The term “between” should be understood as meaning limits included, unless otherwise specified.

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A composition of foundation type comprising, in a physiologically acceptable medium, at least one coloring agent, said composition having a homogenization power 1/ΔE₁ and a covering power 1/ΔE₂ below, when it is applied, according to the value of its lightness L*, to one of the following contrast cards:
when the composition has a lightness L* of between 30 and 40, when it is applied to a contrast card with five zones each respectively having as colorimetric coordinates, to within 15% for L* and h, and to within 25% for C*,

| | | | |
|---|---|---|---|
| first zone (B11+): | L* = 36.7 | C* = 19.81 | h = 47.34°, |
| second zone (B11): | L* = 38.43 | C* = 21.76 | h = 46.51°, |
| third zone (B12): | L* = 35.66 | C* = 19.78 | h = 46.32°, |
| fourth zone (B12+): | L* = 32.98 | C* = 17.29 | h = 44.64°, |
| fifth zone (XXX): | L* = 29.63 | C* = 15.06 | h = 40.34°, |

the composition has a homogenization power 1/ΔE_{1 mean} of between about 1.1 and about 6.7, and a covering power 1/ΔE₂ of between about 0.25 and about 0.85;
when the composition has a lightness of between 40 and 50, when it is applied to a contrast card with five zones each respectively having as calorimetric coordinates, to within 15% for L* and h, and to within 25% for C*,

| | | | |
|---|---|---|---|
| first zone (C9): | L* = 45.04 | C* = 25.18 | h = 53.27°, |
| second zone (B12): | L* = 35.66 | C* = 19.78 | h = 46.32°, |
| third zone (C11): | L* = 38.73 | C* = 21.94 | h = 50.18°, |
| fourth zone (C10): | L* = 42.19 | C* = 24.18 | h = 51.94°, |
| fifth zone (C8): | L* = 48.06 | C* = 25.97 | h = 53.09°, |

the composition has a homogenization power 1/ΔE₁ mean of between about 1.1 and about 1.54 and a covering power 1/ΔE₂ of between about 1/9 and ⅕and better still between about ⅛ and about 0.15,
when the composition has a lightness of between 50 and 60, when it is applied to a contrast card with five zones each respectively having as colorimetric coordinates, to within 15% for L* and h, and to within 25% for C*,

| | | | |
|---|---|---|---|
| first zone (D6): | L* = 54.08 | C* = 26.70 | h = 57.35°, |
| second zone (C11): | L* = 38.73 | C* = 21.94 | h = 50.18°, |
| third zone (D8): | L* = 47.94 | C* = 26.18 | h = 56.82°, |
| fourth zone (D7): | L* = 51.79 | C* = 27.21 | h = 57.09°, |
| fifth zone (D5): | L* = 57.61 | C* = 26.22 | h = 55.09°, |

the composition has a homogenization power 1/ΔE_{1 mean} of between about 0.8 and about 1.25 and a covering power 1/ΔE₂ of between about 1/7 and ⅓.

2. A composition of foundation type comprising, in a cosmetically acceptable medium, at least one coloring agent, said composition having, when it is applied to a contrast card with five zones each respectively having as calorimetric coordinates, to within 5%:

| | | | |
|---|---|---|---|
| first zone (Z1): | L* = 48.38 | a* = 7.99 | b* = 3.85 |
| second zone (Z2): | L* = 46.67 | a* = 6.78 | b* = 3.25 |
| third zone (Z3): | L* = 44.5 | a* = 6.76 | b* = 3.1 |
| fourth zone (Z4): | L* = 42.72 | a* = 4.12 | b* = 2.57 |
| fifth zone (Z5): | L* = 44.41 | a* = 6.57 | b* = 3.93 |

and a sixth zone (Z6) having as calorimetric coordinates

| | | |
|---|---|---|
| L* = 52.26 | a* = 9.11 | b* = 5.81, |

a homogenization power 1/ΔE_{1 mean} of between ¼ and 1, and a covering power 1/ΔE₂ of between 1/25 and 1/7.

3. The composition according to claim 2, said composition of having a homogenization power 1/ΔE₁ mean of between 1/1.6 and ½ and a covering power 1/ΔE₂ of between 1/12 and 1/15.

4. The composition according to claim 2, said composition having a homogenization power 1/ΔE_{1 mean} of between 1/1.8 and 1/2.2 and a covering power 1/ΔE₂ of between 1/13 and 1/17.

5. The composition according to claim 2, said composition having a homogenization power 1/ΔE_{1 mean} of between 1/1.6 and 1/2.1 and a covering power 1/ΔE₂ of between 1/12and 1/16.

6. The composition according to claim 2, said composition having a homogenization power 1/ΔE₁ mean of between 1/2.6 and ⅓ and a covering power 1/ΔE₂ of between 1/16 and 1/21.

7. The composition according to claim 2, said composition having a homogenization power 1/ΔE_{1 mean of between} 1/1.7 and 1/2.2 and a covering power 1/ΔE₂ of between 1/9 and 1/13.

8. The composition according to claim 1, said composition being liquid at room temperature.

9. The composition according to claim 2, said composition being liquid at room temperature.

10. A method for making up a dark skin, comprising applying to the skin a composition as defined in claim 1.

11. A method for making up a dark skin, comprising applying to the skin a composition as defined in claim 2.

12. A method for lightening a dark skin, comprising applying to the skin a composition as defined in claim 1.

13. A method for lightening a dark skin, comprising applying to the skin a composition as defined in claim 2.

14. The composition according to claim 1, wherein the values of L* and h are given with a tolerance selected from the group consisting of: within 12.5%, within 7.5%, within 5%.

15. The composition according to claim 1, wherein the values of C* are given with a tolerance selected from the group consisting of: within 20%, within 15%, within 10%, within 5%.

16. A contrast card comprising at least two colored zones corresponding, respectively, to the mean color of at least two regions of the face of a panel of individuals.

17. The contrast card according to claim 16, said card comprising another colored zone corresponding to the mean color of a region of the body located other than on the face.

18. The contrast card according to claim 16, wherein the colored zones are made so as to have substantially the same color under two different illuminants.

19. The contrast card according to claim 16, said card comprising at least three colored zones corresponding, respectively, to the mean color of the forehead, of a bag under the eyes and of the region between the top lip and the nose of the individuals of the panel.

20. The contrast card according to claim 19, said card also comprising two colored zones corresponding to the color of skin marks of the individuals of the panel.

21. The contrast card according to claim 16, said card also comprising a white zone.

22. The contrast card according to claims 16, said card also comprising a black zone.

23. A method for determining at least one calorimetric characteristic of a composition, comprising the following steps:
coating a contrast card as defined in claims 16, with a coat of a composition,
measuring the color of said zones of the card via the composition, and
determining at least one calorimetric characteristic of the composition (1/ΔE₁; 1/ΔE₂) as a function of color differences measured between the zones.

24. The method according to claim 23, wherein the composition is applied onto a transparent support deposited onto the card.

25. A method for manufacturing a contrast card for evaluating at least one colorimetric characteristic of a composition, comprising the following steps:
selecting a panel of individuals having the same typology of skin:
for each individual of the panel,
measuring the color of at least one region of the body located other than on the face,
measuring the color of at least one region of the face, or
measuring the color of at least two regions of the face,
calculating a mean color for each region, and
reproducing, by printing, the mean colors thus calculated on a contrast card.

26. The method according to claim 25, wherein for at least one individual of the panel, the color of at least three different regions of the face is measured.

27. A method for manufacturing a composition to be applied to skin of a given typology, comprising the following steps:
selecting at least one coloring agent for the composition, using a contrast card as defined in claim 16, and
manufacturing the composition with said coloring agent.

28. A method for marketing the composition as defined in claim 1, wherein, during the marketing of the composition, the color of at least one skin, for which the composition is preferably intended, is taken into account.

29. The method according to claim 28, comprising presenting the color of at least one skin for which the composition is intended.

30. The method according to claim 29, wherein the color is presented with a color indicator on a container or a packaging containing the composition.

31. The method according to claim 28, wherein reference is made on a packaging or a container containing the composition to an ethnic group in which the skin color for which the composition is intended is frequently found.

32. A method for making up dark skin, comprising measuring a colorimetric characteristic of said dark skin, selecting a composition suitable for lightening said dark skin based on the measured colorimetric characteristic, and applying the selected composition to said dark skin.

33. The method according to claim 32, in which the at least one of lightening and saturation of the skin is measured.

34. The composition according to claim 1, wherein when the composition has a lightness of between 30 and 40, the composition has a homogenization power 1/ΔE_{1 mean} of between about 1.25 and about 5.

35. The composition according to claim 1, wherein when the composition has a lightness of between 40 and 50, the composition has a homogenization power 1/ΔE₁ mean of between about 1.1 and about 1.43.

36. The composition according to claim 2, said composition having a homogenization power 1/ΔE₁ mean of between ⅓ and ½.

37. The composition according to claim 1, wherein when the composition has a lightness of between 40 and 50, the composition has a covering power of 1/ΔE₂ mean of between ⅛ and 0.15.

38. The composition according to claim 2, said composition having a covering power 1/ΔE₂ mean of between 1/21 and 1/10.
